(19) United States
(12) Patent Application Publication
(10) Pub. No.: US 2003/0170686 A1
(43) Pub. Date: Sep. 11, 2003

(54) Method and apparatus for washing magnetically responsive particles

(76) Inventors: Rene Hoet, Maastricht (NL); Henricus Renerus Jacobus Mattheus Hoogenboom, Maastricht (NL); Henk Pieters, Maastricht (NL); Robert C. Ladner, Ijamsville, MD (US); Shannon Hogan, Arlington, MA (US); Kristin Rookey, Revere, MA (US)

Correspondence Address: LOUIS MYERS, Fish & Richardson P.C., 225 Franklin Street, Boston, MA, 02110-2804, US

(21) Appl. No.: 10/313,822

(22) Filed: Dec. 6, 2002

Related U.S. Application Data
(60) Provisional application No. 60/337,755, filed on Dec. 7, 2001.
(60) Provisional application No. 60/408,624, filed on Sep. 5, 2002.

(51) Int. Cl. C12Q 1/70; C12Q 1/68; G01N 33/53; G01N 33/567

(52) U.S. Cl. 435/6; 435/5; 435/7.1; 435/7.2

(57) ABSTRACT

The invention features apparati and methods for washing magnetically responsive particles such as paramagnetic beads. One exemplary apparatus includes a flow chamber having an inlet and an outlet; and at least a first magnetic field inducer. The apparatus is configured such that a first magnetic field can be selectively applied in the flow chamber. The apparatus can also apply a flow of fluid to the chamber in coordination with the selective application of the magnetic field.

### CROSS-REFERENCE TO RELATED APPLICATIONS

This application claims benefit of priority to U.S. Provisional Patent Application Serial No. 60/337,755, filed Dec. 7, 2001, and No. 60/408,624, filed Sep. 5, 2002, the contents of both of which are incorporated by reference in their entireties.

### BACKGROUND

The binding of one compound to another is routinely used in isolation methods. For example, many types of chromatography such as ion exchange, affinity, hydrophobic interaction, and immobilized metal chromatographies depend on differential affinity between a compound of interest and other compounds for the chromatography matrix. The separation step, however, frequently requires careful washing of the matrix to remove the other compounds.

Other routinely practiced separations utilize differential sedimentation or precipitation properties. For example, using appropriate solution and centrifugation conditions, a compound of interest can be pelleted from a mixture while other compounds in the mixture remain in the supernatant.

The advent of magnetically responsive beads that can present targets on their surfaces has allowed for separation processes that have many of the advantages of both chromatography and sedimentation. Magnetically responsive beads are typically mixed with a buffer solution in a tube. The tube is agitated in order to effectively wash the beads. The beads are then localized to a magnetic field while buffer solutions are exchanged, for example by aspirating liquid from the tube. Such steps can be faster than centrifugation steps or bench-top sedimentation at 1 g. Like chromatographic matrices, the surfaces of magnetically responsive beads are adaptable for many types of adherent surfaces including immobilized metals, antibodies, and cells.

### SUMMARY

The invention provides, in part, a methodology for efficiently washing and retaining magnetically response particles and an apparatus that selectively applies at least one magnetic field to a flow chamber. The apparatus can be used in combination with the methodology to efficiently wash magnetically responsive particles. Further, both the methodology and the apparatus can be used independently of each other. In some embodiments, they are used (independently or in combination) to isolate a member of a display library for a particular property.

In one aspect, the invention features an apparatus that includes: a flow chamber having an inlet and an outlet; and at least a first magnetic field inducer. The apparatus is configured such that a first and second magnetic field can be selectively applied in the flow chamber. The first and second magnetic fields are spatially distinct. They can be non-overlapping or partially overlapping.

A “flow chamber” refers to a vessel that includes at least an inlet and an outlet such that liquid can flow through the vessel from the inlet to the outlet. The vessel can be sealed but for the inlet and outlet, or open (e.g., the inlet and outlet can be along a lateral face, and an upper surface can be uncovered). The inlet and outlet can be configured such that flow can be controlled and/or monitored, and such that a washing or flow procedure can exchange or replace in a reasonable amount of time at least some (e.g., a large proportion of) fluid in the chamber.

In one preferred embodiment, the apparatus includes a single magnetic field inducer, e.g., a permanent magnet. The magnetic field inducer can be moved from a first position to a second position, thereby applying the first and second magnetic fields. The magnetic field inducer can also be moved, e.g., to a third position, e.g., to apply a third magnetic field.

In another preferred embodiment, the apparatus includes at least a first and second magnetic field inducer. The inducers can be permanent magnets. The first magnetic field inducer can be actuated from a first position that applies a magnetic field within the flow chamber to a second position where it does not apply a magnetic field within the flow chamber. Likewise, the second magnetic field inducer can be actuated from a third position to fourth position in order to selectively apply the second magnetic field. The actuation of the first and second magnetic field inducers can be synchronized, e.g., the first and second magnetic field inducers can be attached to the same actuator. For example, they can be rigidly connected to one another.

In another embodiment, the apparatus further includes at least a third magnetic field inducer that is also controllable. The third magnetic field inducer can apply a third magnetic field.

In a preferred embodiment, the volume of the chamber is at least 0.05, 0.1, 0.2, 0.5, 5, or 50 ml and preferably less then 100, 50, 10, 5, 2, 1, 0.5, 0.3, or 0.2 ml, e.g., between 0.05 and 0.5 ml. In one embodiment, the flow chamber is a cylinder, e.g., a cylinder having an internal diameter of about 0.1 to 5 mm, 3 to 10, or 5 to 20 mm. In a preferred embodiment, the diameter is sufficiently narrow that an aqueous fluid enters by capillary action.

The flow chamber can be composed of, e.g., metal, plastic (e.g., polystyrene or polypropylene or any derivative), or glass. In one embodiment, the flow chamber is non-magnetizeable, e.g., non-metal. In another embodiment, the narrowest cross-section of the flow chamber is rectangular. In a preferred embodiment, the flow chamber is non-horizontal, e.g., vertical. In another embodiment, the flow chamber is non-vertical, e.g., horizontal. In one embodiment, the flow chamber is sterilized or sterilizable (e.g., resistant to treatment with organic solvents or resistant to autoclaving).

Typically the flow chamber is closed so that fluid or air can only enter and exit the system via the inlet and outlet. In one embodiment, the flow chamber includes at least a third port (e.g., a second inlet and/or outlet).

In one embodiment, the apparatus further includes an aqueous solution and a plurality of magnetically responsive particles, e.g., paramagnetic particles, in the flow chamber. A magnetically responsive particle can have an attached target molecule. The attached target molecules can be the same for all the particles. In another embodiment more than one target can be attached to the magnetically responsive particle. For example, two, three, four or more targets are attached. The targets can be related or unrelated. In still another embodiment, only one target is attached to the magnetically responsive particle.

In one embodiment, the target or targets are displayed on the surface of a lipid bilayer that is attached to the magnetically responsive particle. The lipid bilayer can be a liposome or the plasma membrane of a cell, i.e., a cell is attached to the magnetically responsive particle.

The average concentration of the target molecules in the flow chamber can be less than about 1 mM, 1 μM, 100 nM, 10 nM, 1 nm, or 0.1 nM.

In one embodiment, one or more of the magnetic fields is at least about 0.05, 0.1, 0.2, 0.3, 0.5, 0.6, 0.7, 1.0 Tesla and/or no more than about 1.6, 1.5, 1.2, 1.0, 0.7, 0.5, 0.3, 0.2, or 0.1 Tesla. In one embodiment, the magnetic field inducers are permanent magnets. The magnets can be controlled, e.g., by altering the distance between one or more of the magnets and the flow chamber or by otherwise changing the position of a magnetic field inducers with respect to the chamber. For example, the magnets can be controlled by a mechanism that translates the magnets between a first and second position or a mechanism that translates the flow chamber relative to the magnets. In a preferred embodiment, the first and second magnetic field inducers are located at opposite, e.g., diametrically opposite, sides of the flow chamber. In another preferred embodiment, the mechanism translates the magnets on an axis that differs from the direction of fluid flow, e.g., on an axis normal to the direction of fluid flow.

In another embodiment, the magnetic field inducers are electromagnets, e.g., magnets formed by a conductive coil or a superconducting electron path. The electromagnets can be diametrically opposed or arranged as alternating rings. The electrical current is controllable to selectively apply the magnetic field. One preferred electromagnet is a conductive wire coil that surrounds a metal rod. The metal rod can be parallel to the flow path, e.g., adjacent to the flow chamber. The electromagnet is regulated by controlling electric current flowing through the wire coil.

The apparatus can further include a frame attached to the first and second magnetic field inducers. In a preferred embodiment, the frame is translatable between a first and second position. The first and second positions can be about 0.3 to 3 cm or 0.5 to 1 cm apart. In one embodiment, the frame is translatable at least along an axis parallel to a flow chamber diameter. The frame can be attached to a mechanism, which allows the frame to be linearly translated, e.g., cam (e.g., a cam driven by a regulator motor), a drive train, or a piston. In another embodiment, the frame is translated manually. In a preferred embodiment, the frame is attached to the cam by a spindle that is eccentrically positioned relative to the axis of rotation.

In another embodiment, the frame is rotated, thereby moving the first and second magnetic field inducers with respect to the flow chamber.

In a preferred embodiment, the frame comprises first and second plates. The first plate can be attached to a mechanism, which can move (e.g., translate or rotate) the frame. For example, the plate can be attached to a spindle on a cam. For example, the first plate can include a slot (e.g., a recessed groove or an aperture) in which the spindle can slide along a first axis (e.g., vertically). As the cam rotates, the spindle is eccentrically driven, and the first plate is reciprocated along a second axis (e.g., horizontally). The second plate is attached to the magnetic field inducers and to the first plate, e.g., by brackets.

The apparatus can further include a support that has a fitting to accept the flow chamber. The support can also include to a mechanism, which guides movement of the frame, e.g., a track for guiding translation of the frame. For example, wherein the frame comprises first and second plates, the support can include two parallel tracks. Each plate is positioned to translate on one of the tracks.

In another embodiment, the flow chamber is translated relative to the frame, thereby controlling the magnetic field inducers.

The apparatus can further include a fluid reservoir and/or a fluid driver, e.g., a pump. The fluid driver moves fluid from the reservoir into the flow chamber at a controlled rate and/or pressure (e.g., at least 0.01, 0.1, or 0.5 MPa). The fluid driver can be adaptable to regulate the flow of liquid, e.g., at rate of at least about 1 μl, 50 μl, 0.2 ml, 0.3 ml, 0.5 ml, 1 ml, or 2 ml per minute. In one embodiment, the apparatus includes at least two fluid reservoirs.

In one embodiment, the apparatus includes a pump to displace fluid into or out of the flow chamber. In a preferred embodiment, the pump uses positive pressure. In a preferred embodiment, the pump is a peristaltic pump. In another preferred embodiment, the pump is a two-chambered alternating pump. For example, the pump expels liquid from one chamber while replenishing the second chamber. The apparatus can include two two-chambered alternating pumps and a mixing chamber in order to provide controlled mixtures (e.g., gradients) of a first and second solution.

The apparatus can further include a temperature controller. In one embodiment, the flow chamber is enveloped by a jacket connected to a temperature control. In another embodiment, the flow chamber is located within a closet that is connected to a temperature control unit.

In a preferred embodiment, the flow chamber is attached to a fluid line that is in fluid communication with the fluid driver or the fluid reservoir. In a much preferred embodiment, the flow chamber is positioned vertically, and the fluid line is connected to the bottom port, which serves as an inlet. In another much preferred embodiment, the flow chamber is again positioned vertically, and the fluid line is connected to the top port, which serves as an inlet.

The apparatus can further include a feed or effluent line that is attachable to the flow chamber. The line can be tubing, e.g., plastic, Tygon™, or other inert polymeric tubing. The tubing interior wall can be silanized. The line can be disposable.

In a preferred embodiment, the apparatus includes a regulated fluid driver and a controller. The controller coordinates selective application of the first and second magnetic fields with regulation of the fluid driver. In a preferred embodiment, the controller includes a clock or timing mechanism. In another preferred embodiment, the controller includes a processor that is configured or configurable to execute instructions.

In one preferred embodiment, the controller cyclically effects the following: decelerate or arrest the fluid driver, alternate or remove the first magnetic field, increase or apply the second magnetic field, and accelerate or re-activate the fluid driver. The controller can impose a delay between the arrest of the fluid driver and the activation of frame translation and a delay between the termination of frame translation and the re-activation of the fluid driver. The effected actions can further include alternating or removing the second magnetic field. For example, the controller can cyclically effect the following: decelerate or arrest the fluid driver, alternately apply the first and then the second magnetic field for a number of subcycles, and accelerate or re-activate the fluid driver. The controller can be, for example, a set of switches (e.g., a mechanical switch, a timer circuit, or an integrated circuit), an embedded processor, or a programmable-processor (e.g., a computer).

In another preferred embodiment, the controller further includes an interface, e.g., a user interface or an interface to a detector. The interface can detect events (e.g., user commands, user requests, and exceptions). For example, the interface can monitor a detector for a parameter, e.g., to determine if a threshold is exceeded.

The apparatus can further include a physical detector. The physical detector can be adapted to monitor a parameter of fluid at a path position prior to the inlet or after the outlet. The physical detector can monitor and/or record information about light absorbance, e.g., spectrophotometric measurements such as light absorbance (e.g., A₂₆₀, A₂₈₀, A₃₄₀, A₄₈₀, A₅₆₀), light scattering, conductivity, temperature, and pressure. In one embodiment, the physical detector detects air, e.g., a bubble or break in the fluid path. In a preferred embodiment, the spectrophotometer includes an excitation beam and a detector, e.g., perpendicular to the beam and having an optical filter, which can detect fluorescence resulting from the excitation beam.

The apparatus can further include a fraction collector, e.g., adapted to receive fluid from the outlet port and dispense the received fluid into a set of receptacles, e.g., tubes or wells. For example, the fraction collector can include a holder for a microtitre plate. In one embodiment, the apparatus further includes a robotic arm. The receptacle, e.g., a microtitre plate, can be accessible to the robotic arm. In a preferred embodiment, the fraction collector selectively positions the set of receptacles such that fluid emerging from the flow chamber outlet port directly enters one of the receptacles. This configuration avoids the use of plastic or other tubing in the effluent line.

In another aspect, the invention features an apparatus that includes: a flow chamber having a first and second port (e.g., an inlet and outlet); and first and second magnetic field inducers, each magnetic field inducer being controllable to selectively generate a magnetic field in a zone in the flow chamber. The first magnetic field inducer induces a first magnetic field (e.g., in a first zone), the second magnetic field inducer induces a second magnetic field (e.g., in a second zone.) The first and second magnetic fields are spatially distinct, but can be partially overlapping.

The inducers can be permanent magnets. The first magnetic field inducer can be actuated from a first position that applies a magnetic field within the flow chamber to a second position where it does not apply a magnetic field within the flow chamber. Likewise, the second magnetic field inducer can be actuated from a third position to fourth position in order to selectively apply the second magnetic field. The actuation of the first and second magnetic field inducers can be synchronized, e.g., the first and second magnetic field inducers can be attached to the same actuator. For example, they can be rigidly connected to one another.

In another embodiment, the apparatus further includes at least a third magnetic field inducer that is also controllable. The third magnetic field inducer can apply a third magnetic field.

In a preferred embodiment, the volume of the chamber is at least 0.05, 0.1, 0.2, 0.5, 5, or 50 ml and preferably less then 100, 50, 10, 5, 2, 1, 0.5, 0.3, or 0.2 ml, e.g., between 0.05 and 0.5 ml. In one embodiment, the flow chamber is a cylinder, e.g., a cylinder having an internal diameter of about 0.1 to 5 mm, 3 to 10, or 5 to 20 mm. In a preferred embodiment, the diameter is sufficiently narrow that an aqueous fluid enters by capillary action.

The flow chamber can be composed of, e.g., metal, plastic (e.g., polystyrene or polypropylene or any derivative), or glass. In one embodiment, the flow chamber is non-magnetizable, e.g., non-metal. In another embodiment, the narrowest cross-section of the flow chamber is rectangular. In a preferred embodiment, the flow chamber is non-horizontal, e.g., vertical. In another embodiment, the flow chamber is non-vertical, e.g., horizontal. In one embodiment, the flow chamber is sterilized or sterilizable (e.g., resistant to treatment with organic solvents or resistant to autoclaving).

Typically the flow chamber is closed so that fluid or air can only enter and exit the system via the inlet and outlet. In one embodiment, the flow chamber includes at least a third port (e.g., a second inlet and/or outlet).

In one embodiment, the apparatus further includes an aqueous solution and a plurality of magnetically responsive particles, e.g., paramagnetic particles, in the flow chamber. A magnetically responsive particle can have an attached target molecule. The attached target molecules can be the same for all the particles. In another embodiment more than one target can be attached to the magnetically responsive particle. For example, two, three, four or more targets are attached. The targets can be related or unrelated. In still another embodiment, only one target is attached to the magnetically responsive particle.

In one embodiment, the target or targets are displayed on the surface of a lipid bilayer that is attached to the magnetically responsive particle. The lipid bilayer can be a liposome or the plasma membrane of a cell, i.e., a cell is attached to the magnetically responsive particle.

The average concentration of the target molecules in the flow chamber can be less than about 1 mM, 1 μM, 100 nM, 10 nM, 1 nm, or 0.1 nM.

In one embodiment, one or more of the magnetic fields is at least about 0.05, 0.1, 0.2, 0.3, 0.5, 0.6, 0.7, 1.0 Tesla and/or no more than about 1.6, 1.5, 1.2, 1.0, 0.7, 0.5, 0.3, 0.2, or 0.1 Tesla. In one embodiment, the magnetic field inducers are permanent magnets. The magnets can be controlled, e.g., by altering the distance between one or more of the magnets and the flow chamber or by otherwise changing the position of a magnetic field inducers with respect to the chamber. For example, the magnets can be controlled by a mechanism that translates the magnets between a first and second position or a mechanism that translates the flow chamber relative to the magnets. In a preferred embodiment, the first and second magnetic field inducers are located at opposite, e.g., diametrically opposite, sides of the flow chamber. In another preferred embodiment, the mechanism translates the magnets on an axis that differs from the direction of fluid flow, e.g., on an axis normal to the direction of fluid flow.

In another embodiment, the magnetic field inducers are electromagnets, e.g., magnets formed by a conductive coil or a superconducting electron path. The electromagnets can be diametrically opposed or arranged as alternating rings. The electrical current is controllable to selectively apply the magnetic field. One preferred electromagnet is a conductive wire coil that surrounds a metal rod. The metal rod can be parallel to the flow path, e.g., adjacent to the flow chamber. The electromagnet is regulated by controlling electric current flowing through the wire coil.

The apparatus can further include a frame attached to the first and second magnetic field inducers. In a preferred embodiment, the frame is translatable between a first and second position. The first and second positions can be about 0.3 to 3 cm or 0.5 to 1 cm apart. In one embodiment, the frame is translatable at least along an axis parallel to a flow chamber diameter. The frame can be attached to a mechanism, which allows the frame to be linearly translated, e.g., cam (e.g., a cam driven by a regulator motor), a drive train, or a piston. In another embodiment, the frame is translated manually. In a preferred embodiment, the frame is attached to the cam by a spindle that is eccentrically positioned relative to the axis of rotation.

In another embodiment, the frame is rotated, thereby moving the first and second magnetic field inducers with respect to the flow chamber.

In a preferred embodiment, the frame comprises first and second plates. The first plate can be attached to a mechanism, which can move (e.g., translate or rotate) the frame. For example, the plate can be attached to a spindle on a cam. For example, the first plate can include a slot (e.g., a recessed groove or an aperture) in which the spindle can slide along a first axis (e.g., vertically). As the cam rotates, the spindle is eccentrically driven, and the first plate is reciprocated along a second axis (e.g., horizontally). The second plate is attached to the magnetic field inducers and to the first plate, e.g., by brackets.

The apparatus can further include a support that has a fitting to accept the flow chamber. The support can also include to a mechanism, which guides movement of the frame, e.g., a track for guiding translation of the frame. For example, wherein the frame comprises first and second plates, the support can include two parallel tracks. Each plate is positioned to translate on one of the tracks.

In another embodiment, the flow chamber is translated relative to the frame, thereby controlling the magnetic field inducers.

The apparatus can further include a fluid reservoir and/or a fluid driver, e.g., a pump. The fluid driver moves fluid from the reservoir into the flow chamber at a controlled rate and/or pressure (e.g., at least 0.01, 0.1, or 0.5 MPa). The fluid driver can be adaptable to regulate the flow of liquid, e.g., at rate of at least about 1 μl, 50 μl, 0.2 ml, 0.3 ml, 0.5 ml, 1 ml, or 2 ml per minute. In one embodiment, the apparatus includes at least two fluid reservoirs.

In one embodiment, the apparatus includes a pump to displace fluid into or out of the flow chamber. In a preferred embodiment, the pump uses positive pressure. In a preferred embodiment, the pump is a peristaltic pump. In another preferred embodiment, the pump is a two-chambered alternating pump. For example, the pump expels liquid from one chamber while replenishing the second chamber. The apparatus can include two two-chambered alternating pumps and a mixing chamber in order to provide controlled mixtures (e.g., gradients) of a first and second solution.

The apparatus can further include a temperature controller. In one embodiment, the flow chamber is enveloped by a jacket connected to a temperature control. In another embodiment, the flow chamber is located within a closet that is connected to a temperature control unit.

In a preferred embodiment, the flow chamber is attached to a fluid line that is in fluid communication with the fluid driver or the fluid reservoir. In a much preferred embodiment, the flow chamber is positioned vertically, and the fluid line is connected to the bottom port, which serves as an inlet. In another much preferred embodiment, the flow chamber is again positioned vertically, and the fluid line is connected to the top port, which serves as an inlet.

The apparatus can further include a feed or effluent line that is attachable to the flow chamber. The line can be tubing, e.g., plastic, Tygon™, or other inert polymeric tubing. The tubing interior wall can be silanized. The line can be disposable.

In a preferred embodiment, the apparatus includes a regulated fluid driver and a controller. The controller coordinates selective application of the first and second magnetic fields with regulation of the fluid driver. In a preferred embodiment, the controller includes a clock or timing mechanism. In another preferred embodiment, the controller includes a processor that is configured or configurable to execute instructions.

In one preferred embodiment, the controller cyclically effects the following: decelerate or arrest the fluid driver, alternate or remove the first magnetic field, increase or apply the second magnetic field, and accelerate or re-activate the fluid driver. The controller can impose a delay between the arrest of the fluid driver and the activation of frame translation and a delay between the termination of frame translation and the re-activation of the fluid driver. The effected actions can further include alternating or removing the second magnetic field. For example, the controller can cyclically effect the following: decelerate or arrest the fluid driver, alternately apply the first and then the second magnetic field for a number of subcycles, and accelerate or re-activate the fluid driver. The controller can be, for example, a set of switches (e.g., a mechanical switch, a timer circuit, or an integrated circuit), an embedded processor, or a programmable-processor (e.g., a computer).

In another preferred embodiment, the controller further includes an interface, e.g., a user interface or an interface to a detector. The interface can detect events (e.g., user commands, user requests, and exceptions). For example, the interface can monitor a detector for a parameter, e.g., to determine if a threshold is exceeded.

The apparatus can further include a physical detector. The physical detector can be adapted to monitor a parameter of fluid at a path position prior to the inlet or after the outlet. The physical detector can monitor and/or record information about light absorbance, e.g., spectrophotometric measurements such as light absorbance (e.g., A₂₆₀, A₂₈₀, A₃₄₀, A₄₈₀, A₅₆₀), light scattering, conductivity, temperature, and pressure. In one embodiment, the physical detector detects air, e.g., a bubble or break in the fluid path. In a preferred embodiment, the spectrophotometer includes an excitation beam and a detector, e.g., perpendicular to the beam and having an optical filter, which can detect fluorescence resulting from the excitation beam.

The apparatus can further include a fraction collector, e.g., adapted to receive fluid from the outlet port and dispense the received fluid into a set of receptacles, e.g., tubes or wells. For example, the fraction collector can include a holder for a microtitre plate. In one embodiment, the apparatus further includes a robotic arm. The receptacle, e.g., a microtitre plate, can be accessible to the robotic arm. In a preferred embodiment, the fraction collector selectively positions the set of receptacles such that fluid emerging from the flow chamber outlet port directly enters one of the receptacles. This configuration avoids the use of plastic or other tubing in the effluent line.

In still another aspect, the invention features an apparatus that includes: a support having a fitting adapted for mounting a flow chamber; at least a first and a second magnetic field inducer; a translatable frame having attached to the magnetic field inducers; and an actuator that translates the frame in response to a control signal, wherein the translation moves the magnetic field inducers relative to a flow chamber if mounted.

The inducers can be permanent magnets. The first magnetic field inducer can be actuated from a first position that applies a magnetic field within the flow chamber to a second position where it does not apply a magnetic field within the flow chamber. Likewise, the second magnetic field inducer can be actuated from a third position to fourth position in order to selectively apply the second magnetic field. The actuation of the first and second magnetic field inducers can be synchronized, e.g., the first and second magnetic field inducers can be attached to the same actuator. For example, they can be rigidly connected to one another.

In another embodiment, the apparatus further includes at least a third magnetic field inducer that is also controllable. The third magnetic field inducer can apply a third magnetic field.

In a preferred embodiment, the volume of the chamber is at least 0.05, 0.1, 0.2, 0.5, 5, or 50 ml and preferably less then 100, 50, 10, 5, 2, 1, 0.5, 0.3, or 0.2 ml, e.g., between 0.05 and 0.5 ml. In one embodiment, the flow chamber is a cylinder, e.g., a cylinder having an internal diameter of about 0.1 to 5 mm, 3 to 10, or 5 to 20 mm. In a preferred embodiment, the diameter is sufficiently narrow that an aqueous fluid enters by capillary action.

The flow chamber can be composed of, e.g., metal, plastic (e.g., polystyrene or polypropylene or any derivative), or glass. In one embodiment, the flow chamber is non-magnetizable, e.g., non-metal. In another embodiment, the narrowest cross-section of the flow chamber is rectangular. In a preferred embodiment, the flow chamber is non-horizontal, e.g., vertical. In another embodiment, the flow chamber is non-vertical, e.g., horizontal. In one embodiment, the flow chamber is sterilized or sterilizable (e.g., resistant to treatment with organic solvents or resistant to autoclaving).

Typically the flow chamber is closed so that fluid or air can only enter and exit the system via the inlet and outlet. In one embodiment, the flow chamber includes at least a third port (e.g., a second inlet and/or outlet).

In one embodiment, the apparatus further includes an aqueous solution and a plurality of magnetically responsive particles, e.g., paramagnetic particles, in the flow chamber. A magnetically responsive particle can have an attached target molecule. The attached target molecules can be the same for all the particles. In another embodiment more than one target can be attached to the magnetically responsive particle. For example, two, three, four or more targets are attached. The targets can be related or unrelated. In still another embodiment, only one target is attached to the magnetically responsive particle.

In one embodiment, the target or targets are displayed on the surface of a lipid bilayer that is attached to the magnetically responsive particle. The lipid bilayer can be a liposome or the plasma membrane of a cell, i.e., a cell is attached to the magnetically responsive particle.

The average concentration of the target molecules in the flow chamber can be less than about 1 mM, 1 μM, 100 nM, 10 nM, 1 nm, or 0.1 nM.

In one embodiment, one or more of the magnetic fields is at least about 0.05, 0.1, 0.2, 0.3, 0.5, 0.6, 0.7, 1.0 Tesla and/or no more than about 1.6, 1.5, 1.2, 1.0, 0.7, 0.5, 0.3, 0.2, or 0.1 Tesla. In one embodiment, the magnetic field inducers are permanent magnets. The magnets can be controlled, e.g., by altering the distance between one or more of the magnets and the flow chamber or by otherwise changing the position of a magnetic field inducers with respect to the chamber. For example, the magnets can be controlled by a mechanism that translates the magnets between a first and second position or a mechanism that translates the flow chamber relative to the magnets. In a preferred embodiment, the first and second magnetic field inducers are located at opposite, e.g., diametrically opposite, sides of the flow chamber. In another preferred embodiment, the mechanism translates the magnets on an axis that differs from the direction of fluid flow, e.g., on an axis normal to the direction of fluid flow.

In another embodiment, the magnetic field inducers are electromagnets, e.g., magnets formed by a conductive coil or a superconducting electron path. The electromagnets can be diametrically opposed or arranged as alternating rings. The electrical current is controllable to selectively apply the magnetic field. One preferred electromagnet is a conductive wire coil that surrounds a metal rod. The metal rod can be parallel to the flow path, e.g., adjacent to the flow chamber. The electromagnet is regulated by controlling electric current flowing through the wire coil.

In one embodiment, the frame is translatable between a first and second position. The first and second positions can be about 0.3 to 3 cm or 0.5 to 1 cm apart. In one embodiment, the frame is translatable at least along an axis parallel to a flow chamber diameter. The frame can be attached to an actuator, which allows the frame to be linearly translated, e.g., cam (e.g., a cam driven by a regulator motor), a drive train, or a piston. In another embodiment, the frame is translated manually. In a preferred embodiment, the frame is attached to the cam by a spindle that is eccentrically positioned relative to the axis of rotation.

In another embodiment, the frame is rotated, thereby moving the first and second magnetic field inducers with respect to the flow chamber.

In a preferred embodiment, the frame comprises first and second plates. The first plate can be attached to a mechanism, which can move (e.g., translate or rotate) the frame. For example, the plate can be attached to a spindle on a cam. For example, the first plate can include a slot (e.g., a recessed groove or an aperture) in which the spindle can slide along a first axis (e.g., vertically). As the cam rotates, the spindle is eccentrically driven, and the first plate is reciprocated along a second axis (e.g., horizontally). The second plate is attached to the magnetic field inducers and to the first plate, e.g., by brackets.

The support can also include to a mechanism, which guides movement of the frame, e.g., a track for guiding translation of the frame. For example, wherein the frame comprises first and second plates, the support can include two parallel tracks. Each plate is positioned to translate on one of the tracks.

In another embodiment, the flow chamber is translated relative to the frame, thereby controlling the magnetic field inducers.

The apparatus can further include a fluid reservoir and/or a fluid driver, e.g., a pump. The fluid driver moves fluid from the reservoir into the flow chamber at a controlled rate and/or pressure (e.g., at least 0.01, 0.1, or 0.5 MPa). The fluid driver can be adaptable to regulate the flow of liquid, e.g., at rate of at least about 1 μl, 50 μl, 0.2 ml, 0.3 ml, 0.5 ml, 1 ml, or 2 ml per minute. In one embodiment, the apparatus includes at least two fluid reservoirs.

In one embodiment, the apparatus includes a pump to displace fluid into or out of the flow chamber. In a preferred embodiment, the pump uses positive pressure. In a preferred embodiment, the pump is a peristaltic pump. In another preferred embodiment, the pump is a two-chambered alternating pump. For example, the pump expels liquid from one chamber while replenishing the second chamber. The apparatus can include two two-chambered alternating pumps and a mixing chamber in order to provide controlled mixtures (e.g., gradients) of a first and second solution.

The apparatus can further include a temperature controller. In one embodiment, the flow chamber is enveloped by a jacket connected to a temperature control. In another embodiment, the flow chamber is located within a closet that is connected to a temperature control unit.

In a preferred embodiment, the flow chamber is attached to a fluid line that is in fluid communication with the fluid driver or the fluid reservoir. In a much preferred embodiment, the flow chamber is positioned vertically, and the fluid line is connected to the bottom port, which serves as an inlet. In another much preferred embodiment, the flow chamber is again positioned vertically, and the fluid line is connected to the top port, which serves as an inlet.

The apparatus can further include a feed or effluent line that is attachable to the flow chamber. The line can be tubing, e.g., plastic, Tygon™, or other inert polymeric tubing. The tubing interior wall can be silanized. The line can be disposable.

In a preferred embodiment, the apparatus includes a regulated fluid driver and a controller. The controller coordinates selective application of the first and second magnetic fields with regulation of the fluid driver. In a preferred embodiment, the controller includes a clock or timing mechanism. In another preferred embodiment, the controller includes a processor that is configured or configurable to execute instructions.

In one preferred embodiment, the controller cyclically effects the following: decelerate or arrest the fluid driver, alternate or remove the first magnetic field, increase or apply the second magnetic field, and accelerate or re-activate the fluid driver. The controller can impose a delay between the arrest of the fluid driver and the activation of frame translation and a delay between the termination of frame translation and the re-activation of the fluid driver. The effected actions can further include alternating or removing the second magnetic field. For example, the controller can cyclically effect the following: decelerate or arrest the fluid driver, alternately apply the first and then the second magnetic field for a number of subcycles, and accelerate or re-activate the fluid driver. The controller can be, for example, a set of switches (e.g., a mechanical switch, a timer circuit, or an integrated circuit), an embedded processor, or a programmable-processor (e.g., a computer).

In another preferred embodiment, the controller further includes an interface, e.g., a user interface or an interface to a detector. The interface can detect events (e.g., user commands, user requests, and exceptions). For example, the interface can monitor a detector for a parameter, e.g., to determine if a threshold is exceeded.

The apparatus can further include a physical detector. The physical detector can be adapted to monitor a parameter of fluid at a path position prior to the inlet or after the outlet. The physical detector can monitor and/or record information about light absorbance, e.g., spectrophotometric measurements such as light absorbance (e.g., A₂₆₀, A₂₈₀, A₃₄₀, A₄₈₀, A₅₆₀), light scattering, conductivity, temperature, and pressure. In one embodiment, the physical detector detects air, e.g., a bubble or break in the fluid path. In a preferred embodiment, the spectrophotometer includes an excitation beam and a detector, e.g., perpendicular to the beam and having an optical filter, which can detect fluorescence resulting from the excitation beam.

The apparatus can further include a fraction collector, e.g., adapted to receive fluid from the outlet port and dispense the received fluid into a set of receptacles, e.g., tubes or wells. For example, the fraction collector can include a holder for a microtitre plate. In one embodiment, the apparatus further includes a robotic arm. The receptacle, e.g., a microtitre plate, can be accessible to the robotic arm. In a preferred embodiment, the fraction collector selectively positions the set of receptacles such that fluid emerging from the flow chamber outlet port directly enters one of the receptacles. This configuration avoids the use of plastic or other tubing in the effluent line.

In a preferred embodiment, the support has a plurality of fittings, each adapted for mounting a flow chamber. The apparatus includes at least a third magnetic field inducer attached to the frame. The magnetic field inducers can be spaced at regular intervals on the frame. In a preferred embodiment, the intervals are at least 1.5 times the diameter of the flow chamber in length. In a preferred embodiment, the North poles of all the magnetic field inducers face the same direction.

In one embodiment, the frame is translatable on an axis substantially perpendicular to the flow path of a mounted flow chamber. In one embodiment, the apparatus further includes a flow chamber. In one embodiment, the apparatus includes a second support, e.g., parallel to the first support. The supports can be planar shelves.

In another aspect, the invention features a system that includes: an apparatus described herein, e.g., having a flow chamber and at least a first magnetic field inducer; a fluid driver, e.g., in fluid communication with the flow chamber; and a controller configured to coordinate selective application of a first and second magnetic fields in the flow chamber with regulation of the fluid driver.

In a preferred embodiment, the controller includes an interface for communication with the apparatus. The controller can also include an interface for communication with the fluid control unit. The communication can be electrical, optical, or wireless. For 1 example, the controller, apparatus and fluid driver can be connected by a data exchange network.

In another preferred embodiment, the controller includes an information storage medium. The controller can be further configured to store instructions, detected parameters, or events.

In another embodiment, the system includes a robotic arm or robot. The robot can dispose magnetically responsive particles in a flow chamber and assemble the flow chamber into the apparatus. The robot can also make necessary fluid connections or other manipulations, e.g., prior to, during, or after one or more steps effected by the controller.

In still another embodiment, the system includes a sample tracking detector, e.g., a bar code scanner or transponder system. Pre-assembled flow chambers can be marked and tracked on insertion into an apparatus and upon removal from the apparatus. Collection vessels, e.g., microtitre plates, can be marked and tracked. Information regarding tracking events can be communicated to the controller or a server.

In another aspect, the invention features a system that includes: an apparatus described herein; a fluid control unit; a machine comprising a processor configured to execute instructions. The instructions cause the processor to: detect a user command; and send\ controls to the apparatus and fluid control unit to effect for one or more cycles of (1) activating flow of liquid by triggering the fluid control unit; (2) optionally, after an interval, arresting flow of the liquid; and (3) activating the apparatus to agitate magnetically responsive particles by alternately applying at least a first and second magnetic field.

The apparatus can be activated to agitate the magnetically responsive particles by controlling the application of the magnetic fields, e.g., by controlling the magnetic field inducers. In a preferred embodiment, the instructions further include monitoring user commands, a pre-set sequence, or operation parameters for a condition to halt the cycle.

In one embodiment, processor monitors a pre-set sequence, e.g., a sequence of timed events. In another embodiment, the processor monitors user commands to detect a user's control of a pointer (e.g., a cursor) on a graphical user interface. In still another embodiment, the processor monitors operation parameters such as back-pressure, A260, and conductivity to determine whether to halt the cycle.

In still another aspect, the invention features an article that includes a machine-readable medium that stores machine-executable instructions (e.g., software). The instructions cause a machine to activate an apparatus to selectively apply a first and second magnetic field to a flow chamber. In one embodiment, a third magnetic field is applied.

In a preferred embodiment, the instructions cause the machine to effect one or more cycles of (1) activating flow of liquid by triggering the fluid control unit; (2) optionally, after an interval, arresting flow of the liquid; (3) activating the apparatus to agitate magnetically responsive particles by alternately selectively applying the first and second magnetic field. In other preferred embodiments, the instructions cause a machine to effect a method described herein, e.g., hereinafter.

The invention also features a system that enables the automated washing of paramagnetic particles in a flow chamber. The system controls movement of particles in the chamber and the flow of liquid through the flow chamber. The particles are moved between at least a first and second zone within the flow chamber.

In one aspect, the invention features a method of washing magnetically responsive particles. The method includes: flowing liquid through a flow chamber that includes magnetically responsive particles captured in a first zone by a first magnetic field; dissipating the first magnetic field; and agitating the magnetically responsive particles. The agitating can be a result of the dissipating.

In one embodiment, the method further includes: reducing or arresting the liquid flow prior to the dissipating. In one example, the flow is not arrested, but attenuated, e.g., decreased to a rate less than 50, 40, 30, 25, 20, 10, 5, 4, 3, 1, or 0.1% of the original flow rate. In still anther embodiment, the flow is not arrest to any extent. Liquid flow can be reduced or arrested prior to removing the first magnetic field, and restored after the agitating. There can be a delay period between the reducing or arresting and the dissipating, e.g., such that liquid is not flowed and the first magnetic field is held in place. The delay can provide time for pressure or disturbances caused by the flow to dissipate. In a preferred embodiment, the method further includes repeating the flowing through the agitating one or more times. For example, the agitating is effected by cyclically re-applying the first magnetic field. The magnetically responsive particles are retained in the flow chamber throughout the flowing, dissipating, and agitating. In one embodiment, the particles are, for example, displaced in a substantially perpendicular direction relative to the direction of solution flow.

In another embodiment, the agitating is effected by applying a second magnetic field, e.g., to capture the magnetically responsive particles in a second zone. In another embodiment, the second magnetic field is applied prior to removing the first magnetic field. In still another embodiment, the second magnetic field is applied while removing the first magnetic field, e.g., in tandem. In another embodiment, the method includes reducing or arresting the flow prior to or during the removal of the first magnetic field, and resuming the flowing after the second magnetic field is applied.

The agitating can include cyclically and alternatively applying the second and first magnetic field. In one embodiment, the first and second magnetic field are applied by first and second permanent magnets that are coordinately regulated. In another embodiment, the agitating includes vibrating or sonicating the flow chamber. In still another, embodiment, the agitating includes generating fluid turbulence or fluid stirring within the flow chamber. Generally, agitation and magnetic field application is configured to prevent the particles from settling to lower regions of the chamber.

The flow chamber can be composed of, e.g., metal, plastic (e.g., polystyrene or polypropylene or any derivative), or glass. In one embodiment, the flow chamber is non-magnetizable, e.g., non-metal. In another embodiment, the narrowest cross-section of the flow chamber is rectangular. In a preferred embodiment, the flow chamber is non-horizontal, e.g., vertical. In another embodiment, the flow chamber is non-vertical, e.g., horizontal. In one embodiment, the flow chamber is sterilized or sterilizable (e.g., resistant to treatment with organic solvents or resistant to autoclaving). In one embodiment, the flow chamber is a fermentor.

In a preferred embodiment, the volume of the chamber is at least 0.05, 0.1, 0.2, 0.5, 5, or 50 ml and preferably less then 100, 50, 10, 5, 2, 1, 0.5, 0.3, or 0.2 ml, e.g., between 0.05 and 0.5 ml. In one embodiment, the flow chamber is a cylinder, e.g., a cylinder having an internal diameter of about 0.1 to 5 mm, 3 to 10, or 5 to 20 mm. In a preferred embodiment, the diameter is sufficiently narrow that an aqueous fluid enters by capillary action.

The first and second magnets can be rigidly attached, e.g., to a frame, which is translated between a first and second position to alternatively apply the first and second magnetic fields. In one embodiment, the flow is controlled by a fluid driver and dissipating the first magnetic field is controlled by a regulator in signal communication with the fluid driver. In another embodiment, the flow is controlled by a fluid driver and dissipating the first magnetic field is controlled by a regulator, and the fluid driver and the regulator are controlled by a programmable processor.

In one embodiment, the magnetically responsive particles include a molecule such as a biomolecule on at an interior or exterior position of the particle. The molecule can be a target molecule or attachable to a target molecule.

The particles can include a target molecule (e.g., a nucleic acid, protein, polysaccharide, etc.) that is covalently or non-covalently attached. In one embodiment, the magnetically responsive particles are bound by a display library member (e.g., a cell, a bacteriophage, an RNA-polypeptide fusion). In one embodiment, the target includes a cell, e.g., a living cell. In another embodiment, the target includes a purified polypeptide, e.g., a human polypeptide or a pathogenic polypeptide. In some cases, the purified polypeptide is an extracellular protein. The protein can include a post-translational modification (e.g., a phosphorylation, proteolytic cleavage, ubiquitination, methylation, or acylation.).

For example, the magnetically responsive particles can be bound by a first compound to a differential extent relative to a second compound, such that under particular conditions, the second compound can be separated from the first compound. e.g., by a wash with a wash solution. The wash solution can include, e.g., a medium for cell growth, a detergent, or a competing agent that competes with the target for binding of the first compound. The method can include isolating the first compound from the second compound. The method can also include analyzing the first or second compound, e.g., by one or more of nucleic acid sequencing, protein sequencing, mass spectroscopy, amplification, surface plasmon resonance, or fluorimetry. The first and second compounds can include polypeptides, e.g., displayed as display library members. The first and second compounds can include non-identical polypeptides that have amino acid sequences that are at least 50, 60, 80, 90, 95, 97, 98, or 99% identical.

In another preferred embodiment, the flow chamber is non-horizontal, e.g., substantially vertical or vertical. The liquid is flowed upwards. The liquid can be a solution (e.g., a wash solution) into which a compound non-specifically bound to the magnetically responsive particle dissociates from the magnetically responsive particle. In still another preferred embodiment, the liquid is flowed downwards. The liquid can be a solution (e.g., an elution solution) into which a compound specifically bound to the magnetically responsive particle dissociates from the magnetically responsive particle.

In one embodiment, the method further includes after (c), (d) removing the second magnetic field and applying the first magnetic field. The method can include 1, 2, 3, 4, 5, 7, 10, 12 or more cycles of (b), (c), and (d). The magnetically responsive particles move from a first to a second zone. The first and second magnetic fields and/or the first and second zones have distinct locations within the flow chamber, yet may be overlapping.

In one embodiment, the flow is at least about 0.01, 0.02, 0.05, 0.1, 0.2, or 0.5 cm/min and/or no more than 0.7, 0.5, 0.2, 0.1, 0.05, or 0.02 cm/min.

In a preferred embodiment, the method is at least partially effected by a machine (i.e. a controller). In a much preferred embodiment, the method is automated. In another embodiment, the method is at least partially effected manually.

In a preferred embodiment, the method is practiced with an apparatus described herein.

In another aspect, the invention features a method that includes: providing a flow chamber, a first magnetic field inducer, and magnetically responsive particles disposed in the chamber, at least some of the particles are physically associated with a target (or attachable to a target); capturing the magnetically responsive particles in a first magnetic field applied by the first magnetic field inducer in the flow chamber; flowing a solution through the flow chamber; and displacing the magnetically responsive particles from the first zone of the flow chamber to other regions of the flow chamber such that the magnetically responsive particles are retained in the flow chamber.

The displacing can include altering the position of the first magnetic field inducer relative to the flow chamber. In one embodiment, the magnetically responsive particles are displaced to a second zone. For example, the displacing can include applying a second magnetic field to displace the magnetically responsive particles to the second zone.

In one embodiment, the method further includes: reducing or arresting the liquid flow prior to the displacing. In one example, the flow is not arrested, but attenuated, e.g., decreased to a rate less than 50, 40, 30, 25, 20, 10, 5, 4, 3, 1, or 0.1% of the original flow rate. In still anther embodiment, the flow is not arrest to any extent. Liquid flow can be reduced or arrested prior to removing the first magnetic field, and restored after the agitating. There can be a delay period between the reducing or arresting and the dissipating, e.g., such that liquid is not flowed and the first magnetic field is held in place. The delay can provide time for pressure or disturbances caused by the flow to dissipate.

In a preferred embodiment, the method further includes repeating the flowing through the agitating one or more times. For example, the agitating is effected by cyclically re-applying the first magnetic field. The magnetically responsive particles are retained in the flow chamber throughout the flowing, dissipating, and agitating. In one embodiment, the particles are, for example, displaced in a substantially perpendicular direction relative to the direction of solution flow.

In another embodiment, the agitating is effected by applying a second magnetic field, e.g., to capture the magnetically responsive particles in a second zone. In another embodiment, the second magnetic field is applied prior to displacing the particles. In still another embodiment, the second magnetic field is applied while removing the first magnetic field, e.g., in tandem. In another embodiment, the method includes reducing or arresting the flow prior to or during the removal of the first magnetic field, and resuming the flowing after the second magnetic field is applied. The first and/or second magnetic field inducers can be permanent magnets or electromagnets.

The agitating can include cyclically and alternatively applying the second and first magnetic field. The first and second magnetic field are applied by first and second permanent magnets that are coordinately regulated. In another embodiment, the agitating includes vibrating or sonicating the flow chamber. In still another, embodiment, the agitating includes generating fluid turbulence or fluid stirring within the flow chamber.

The flow chamber can be composed of, e.g., metal, plastic (e.g., polystyrene or polypropylene or any derivative), or glass. In one embodiment, the flow chamber is non-magnetizable, e.g., non-metal. In another embodiment, the narrowest cross-section of the flow chamber is rectangular. In a preferred embodiment, the flow chamber is non-horizontal, e.g., vertical. In another embodiment, the flow chamber is non-vertical, e.g., horizontal. In one embodiment, the flow chamber is sterilized or sterilizable (e.g., resistant to treatment with organic solvents or resistant to autoclaving). In one embodiment, the flow chamber is a fermentor.

In a preferred embodiment, the volume of the chamber is at least 0.05, 0.1, 0.2, 0.5, 5, or 50 ml and preferably less then 100, 50, 10, 5, 2, 1, 0.5, 0.3, or 0.2 ml, e.g., between 0.05 and 0.5 ml. In one embodiment, the flow chamber is a cylinder, e.g., a cylinder having an internal diameter of about 0.1 to 5 mm, 3 to 10, or 5 to 20 mm. In a preferred embodiment, the diameter is sufficiently narrow that an aqueous fluid enters by capillary action.

The first and second magnets can be rigidly attached, e.g., to a frame, which is translated between a first and second position to alternatively apply the first and second magnetic fields. The flow of the liquid can be arrested prior to translating the frame to the second position. The flow can be resumed after the frame is translated. The flow chamber can be supported by a first and optionally a second shelf. The frame can translate along a recessed track in the first and/or second shelf. The frame can be translated by a cam that is controlled by a motor. In one embodiment, the flow is controlled by a fluid driver and dissipating the first magnetic field is controlled by a regulator in signal communication with the fluid driver. In another embodiment, the flow is controlled by a fluid driver and dissipating the first magnetic field is controlled by a regulator, and the fluid driver and the regulator are controlled by a programmable processor.

In one embodiment, the magnetically responsive particles include a molecule such as a biomolecule on at an interior or exterior position of the particle. The molecule can be a target molecule or attachable to a target molecule.

The particles include a target molecule (e.g., a nucleic acid, protein, polysaccharide, etc.) that is covalently or non-covalently attached. In one embodiment, the magnetically responsive particles are bound by a display library member (e.g., a cell, a bacteriophage, an RNA-polypeptide fusion). In one embodiment, the target includes a cell, e.g., a living cell. In another embodiment, the target includes a purified polypeptide, e.g., a human polypeptide or a pathogenic polypeptide. In some cases, the purified polypeptide is an extracellular protein. The protein can include a post-translational modification (e.g., a phosphorylation, proteolytic cleavage, ubiquitination, methylation, or acylation.).

For example, the magnetically responsive particles can be bound by a first compound to a differential extent relative to a second compound, such that under particular conditions, the second compound can be separated from the first compound. e.g., by a wash with a wash solution. The wash solution can include, e.g., a medium for cell growth, a detergent, or a competing agent that competes with the target for binding of the first compound. The method can include isolating the first compound from the second compound. The method can also include analyzing the first or second compound, e.g., by one or more of nucleic acid sequencing, protein sequencing, mass spectroscopy, amplification, surface plasmon resonance, or fluorimetry. The first and second compounds can include polypeptides, e.g., displayed as display library members. The first and second compounds can include non-identical polypeptides that have amino acid sequences that are at least 50, 60, 80, 90, 95, 97, 98, or 99% identical.

In another preferred embodiment, the flow chamber is non-horizontal, e.g., substantially vertical or vertical. The liquid is flowed upwards. The liquid can be a solution (e.g., a wash solution) into which a compound non-specifically bound to the magnetically responsive particle dissociates from the magnetically responsive particle. In still another preferred embodiment, the liquid is flowed downwards. The liquid can be a solution (e.g., an elution solution) into which a compound specifically bound to the magnetically responsive particle dissociates from the magnetically responsive particle.

In one embodiment, the method further includes after (c), (d) removing the second magnetic field and applying the first magnetic field. The method can include 1, 2, 3, 4, 5, 7, 10, 12 or more cycles of (b), (c), and (d). The magnetically responsive particles move from a first to a second zone. The first and second magnetic fields and/or the first and second zones have distinct locations within the flow chamber, yet may be overlapping.

In one embodiment, the flow is at least about 0.01, 0.02, 0.05, 0.1, 0.2, or 0.5 cm/min and/or no more than 0.7, 0.5, 0.2, 0.1, 0.05, or 0.02 cm/min.

In a preferred embodiment, the method is at least partially effected by a machine (i.e. a controller). In a much preferred embodiment, the method is automated. In another embodiment, the method is at least partially effected manually.

In a preferred embodiment, the method is practiced with an apparatus described herein

In another aspect, the invention features a method that includes: providing a flow chamber, at least a first magnetic field inducer, and magnetically responsive particles disposed in the chamber; capturing the magnetically responsive particles in a first zone in the flow chamber; flowing a solution through the flow chamber; and displacing the magnetically responsive particles from the first zone to a second zone in the flow chamber. At least some of the particles are physically associated (or associable) with a target, e.g., covalently bound or non-covalently bound to the target.

In one embodiment, each particle is associated with the same target. In another embodiment, at least some of the particles are associated with different targets. The targets can be covalently attached to the particles, or non-covalently attached. The targets can be attached, e.g., by a bridge, e.g., formed by a binding pair, e.g. a specific binding pair. The particles can be associated with the target, before, during or after the capturing and/or the flowing.

The magnetically responsive particles can be about 1 to 10 μm in diameter, e.g., about 2 to 5 μm in diameter. They can have modified surfaces (external or internal surfaces) are physically associated with the target. Exemplary targets include molecules such as haptens, transition state analogs, and proteins. The target can be, for example, a multimeric protein complex. Other exemplary targets include cells, e.g., cancer cells, transformed cells, blood cells, bacterial cells, fungal cells, and plant cells. Exemplary members of specific binding pairs include immobilized metals (e.g., bound by hexa-histidine), biotin (e.g., bound by avidin), other small organic molecules, and antibodies (e.g., bound by respective antigens).

In a preferred embodiment, cells are indirectly bound to the magnetically responsive particles, e.g., by an antibody, e.g., an antibody to a marker. The marker can be specific for the cell, e.g., present on a cancer cell, but not a normal cell.

The target can be bound by a first and second compound that have a differential affinity, specificity, or avidity for the target. For example, the flowing can be under conditions such that the second compound is preferentially washed from the target (e.g., target bound to a particle) relative to the first compound. The method can include separating the first and second compounds. In one embodiment, the first and second compounds include nucleic acids, proteins, carbohydrates, metabolites, cells, or viruses. In a preferred embodiment, the first and second compounds are display entities, e.g., in a virus (e.g., phage), cell, mRNA-protein fusion, or ribosome display format (e.g., as described herein).

The method can further include analyzing the first compound and/or the second compound. The analysis can comprise, e.g., amplification (e.g., nucleic acid amplification, cloning, or propagation (e.g., for cells and viruses)), nucleic acid hybridization (e.g., in solution, or to a support such as an array), mass-spectrometry, surface plasmon resonance, optical monitoring (e.g., fluorimetry), and a biological activity assay.

The first or second compound can be processed through any number of additional steps prior to analysis. For example, the first or second compound can be a bacteriophage. The bacteriophage can infected into cells, propagated, processed to isolate bacteriophage nucleic acid, and sequenced. A relevant fragment of the bacteriophage nucleic acid can be subcloned and expressed, e.g., in a vector, or the sequence of the fragment can be interpreted and used to instruct the chemical synthesis of a peptide or new nucleic acid.

In one embodiment, the first and second compound differ in binding affinity for the target by less than 5, 4, 3, or 2 orders of magnitude. In a preferred embodiment, the first and second compounds are nucleic acids or polypeptide that are at least 50% identical (e.g., at least 55, 60, 70, 80, 85, 90, 95, or 99% identical). The first and second compound can be identical in one or more segments. For polypeptide compounds, the first and second compound can have identical or non-identical scaffold structures. The first and/or second compounds are members of a library, e.g., a display library.

The displacing can include altering the position of the first magnetic field inducer relative to the flow chamber. In one embodiment, the magnetically responsive particles are displaced to a second zone. For example, the displacing can include applying a second magnetic field to displace the magnetically responsive particles to the second zone. In a preferred embodiment, the magnetic field is removed from the first zone, e.g., prior to, during or after, applying the magnetic field to the second zone.

In one embodiment, the method further includes: reducing or arresting the liquid flow prior to the displacing. In one example, the flow is not arrested, but attenuated, e.g., decreased to a rate less than 50, 40, 30, 25, 20, 10, 5, 4, 3, 1, or 0.1% of the original flow rate. In still anther embodiment, the flow is not arrest to any extent. Liquid flow can be reduced or arrested prior to removing the first magnetic field, and restored after the agitating. There can be a delay period between the reducing or arresting and the dissipating, e.g., such that liquid is not flowed and the first magnetic field is held in place. The delay can provide time for pressure or disturbances caused by the flow to dissipate.

In a preferred embodiment, the method further includes repeating the flowing through the agitating one or more times. For example, the agitating is effected by cyclically re-applying the first magnetic field. The magnetically responsive particles are retained in the flow chamber throughout the flowing, dissipating, and agitating. In one embodiment, the particles are, for example, displaced in a substantially perpendicular direction relative to the direction of solution flow.

In another embodiment, the agitating is effected by applying a second magnetic field, e.g., to capture the magnetically responsive particles in a second zone. In another embodiment, the second magnetic field is applied prior to displacing the particles. In still another embodiment, the second magnetic field is applied while removing the first magnetic field, e.g., in tandem. In another embodiment, the method includes reducing or arresting the flow prior to or during the removal of the first magnetic field, and resuming the flowing after the second magnetic field is applied. The first and/or second magnetic field inducers can be permanent magnets or electromagnets.

The agitating can include cyclically and alternatively applying the second and first magnetic field. In one embodiment, the first and second magnetic field are applied by first and second permanent magnets that are coordinately regulated. In another embodiment, the agitating includes vibrating or sonicating the flow chamber. In still another, embodiment, the agitating includes generating fluid turbulence or fluid stirring within the flow chamber.

The flow chamber can be composed of, e.g., metal, plastic (e.g., polystyrene or polypropylene or any derivative), or glass. In one embodiment, the flow chamber is non-magnetizable, e.g., non-metal. In another embodiment, the narrowest cross-section of the flow chamber is rectangular. In a preferred embodiment, the flow chamber is non-horizontal, e.g., vertical. In another embodiment, the flow chamber is non-vertical, e.g., horizontal. In one embodiment, the flow chamber is sterilized or sterilizable (e.g., resistant to treatment with organic solvents or resistant to autoclaving). In one embodiment, the flow chamber is a fermentor.

In a preferred embodiment, the volume of the chamber is at least 0.05, 0.1, 0.2, 0.5, 5, or 50 ml and preferably less then 100, 50, 10, 5, 2, 1, 0.5, 0.3, or 0.2 ml, e.g., between 0.05 and 0.5 ml. In one embodiment, the flow chamber is a cylinder, e.g., a cylinder having an internal diameter of about 0.1 to 5 mm, 3 to 10, or 5 to 20 mm. In a preferred embodiment, the diameter is sufficiently narrow that an aqueous fluid enters by capillary action.

The first and second magnets can be rigidly attached, e.g., to a frame, which is translated between a first and second position to alternatively apply the first and second magnetic fields. The flow of the liquid can be arrested prior to translating the frame to the second position. The flow can be resumed after the frame is translated. The flow chamber can be supported by a first and optionally a second shelf. The frame can translate along a recessed track in the first and/or second shelf. The frame can be translated by a cam that is controlled by a motor. In one embodiment, the flow is controlled by a fluid driver and dissipating the first magnetic field is controlled by a regulator in signal communication with the fluid driver. In another embodiment, the flow is controlled by a fluid driver and dissipating the first magnetic field is controlled by a regulator, and the fluid driver and the regulator are controlled by a programmable processor.

In one embodiment, the magnetically responsive particles include a molecule such as a biomolecule on at an interior or exterior position of the particle. The molecule can be a target molecule or attachable to a target molecule.

The particles include a target molecule (e.g., a nucleic acid, protein, polysaccharide, etc.) that is covalently or non-covalently attached. In one embodiment, the magnetically responsive particles are bound by a display library member (e.g., a cell, a bacteriophage, an RNA-polypeptide fusion). In one embodiment, the target includes a cell, e.g., a living cell. In another embodiment, the target includes a purified polypeptide, e.g., a human polypeptide or a pathogenic polypeptide. In some cases, the purified polypeptide is an extracellular protein. The protein can include a post-translational modification (e.g., a phosphorylation, proteolytic cleavage, ubiquitination, methylation, or acylation.).

For example, the magnetically responsive particles can be bound by a first compound to a differential extent relative to a second compound, such that under particular conditions, the second compound can be separated from the first compound. e.g., by a wash with a wash solution. The wash solution can include, e.g., a medium for cell growth, a detergent, or a competing agent that competes with the target for binding of the first compound. The method can include isolating the first compound from the second compound. The method can also include analyzing the first or second compound, e.g., by one or more of nucleic acid sequencing, protein sequencing, mass spectroscopy, amplification, surface plasmon resonance, or fluorimetry. The first and second compounds can include polypeptides, e.g., displayed as display library members. The first and second compounds can include non-identical polypeptides that have amino acid sequences that are at least 50, 60, 80, 90, 95, 97, 98, or 99% identical.

In another preferred embodiment, the flow chamber is non-horizontal, e.g., substantially vertical or vertical. The liquid is flowed upwards. The liquid can be a solution (e.g., a wash solution) into which a compound non-specifically bound to the magnetically responsive particle dissociates from the magnetically responsive particle. In still another preferred embodiment, the liquid is flowed downwards. The liquid can be a solution (e.g., an elution solution) into which a compound specifically bound to the magnetically responsive particle dissociates from the magnetically responsive particle.

In one embodiment, the method further includes after (c), (d) removing the second magnetic field and applying the first magnetic field. The method can include 1, 2, 3, 4, 5, 7, 10, 12 or more cycles of (b), (c), and (d). The magnetically responsive particles move from a first to a second zone. The first and second magnetic fields and/or the first and second zones have distinct locations within the flow chamber, yet may be overlapping.

In one embodiment, the flow is at least about 0.01, 0.02, 0.05, 0.1, 0.2, or 0.5 cm/min and/or no more than 0.7, 0.5, 0.2, 0.1, 0.05, or 0.02 cm/min.

In a preferred embodiment, the method is at least partially effected by a machine (i.e. a controller). In a much preferred embodiment, the method is automated. In another embodiment, the method is at least partially effected manually.

In a preferred embodiment, the method is practiced with an apparatus described herein

In another aspect, the invention features a method that includes: a) disposing magnetically responsive particles in a flow chamber; b) contacting the magnetically responsive particles to a mixture; c) applying a first magnetic field to the flow chamber; d) flowing liquid through the chamber; and e) agitating the magnetically responsive particles by alternatively applying the first magnetic field and the second magnetic field. The magnetically responsive particles have a target attached to their surface.

The order need not be sequential. For example, a) can be effected prior to b), c) prior to b), and so forth. In a preferred embodiment, the steps are performed in sequential order.

In a preferred embodiment, the mixture is a display library, e.g., a display library described herein such as a phage, cell, mRNA-protein fusion, or ribosome display library. The library can have a high diversity, e.g., at least 10⁶, 10⁷, 10⁸, 10⁹, 10¹⁰ members, or a limited diversity, e.g., no more than 10⁶, 10⁵, 10⁴, or 10³ members.

In another embodiment, the mixture includes nucleic acids, proteins, carbohydrates, or metabolites. In still another embodiment, the mixture includes a cell extract, tissue homogenate, serum, or cell population, e.g., a population of blood cells, dissociated tissue cells, or microorganisms (e.g., bacterial cells and fungal cells).

In one embodiment, the method further includes determining a qualitative or quantitative measure of the number members of the complex mixture that are bound or are not bound to the particles after d) and/or e).

In a preferred embodiment, the method further includes (f) analyzing a subset of the complex mixture, e.g., a subset that dissociates from the target or that is not bound by the target, or a subset that binds the target.

The liquid can be an aqueous buffer. For example, the liquid can be a low salt buffer having an ionic strength of less than about 150 mM, e.g., a physiological buffer such as PBS. The buffer can include a pH stabilizing buffering agent such as Tris, HEPES, or another buffer. The pH of the buffer can be in the range of 5 to 10, 6 to 9, or 6.5 to 8.5. The liquid can be a high salt solution, e.g., having an ionic strength of at least 200 mM. The buffer can include a reducing reagent, e.g., dithiothreitol (DTT) or β-mercaptoethanol. The buffer can include a detergent, e.g., sodium dodecyl sulfate (SDS) (e.g., in amount of less than 0.1%), Triton-X-100, Tween-20, and so forth. In another embodiment, the liquid includes an acid or a base, e.g., the liquid has a pH of less than 6, 5, 4, or 3.5 or the liquid has a pH of at least 8, 9, 10, 10.5, or 11. In still another embodiment, the liquid includes a chaotrope, e.g., guanidine or urea.

The method can, of course, be practiced with an apparatus described herein and/or with any selection of features described herein.

In another aspect, the invention features a method of washing magnetically responsive particles. The method includes: (1) contacting magnetically responsive particles with a sample; (2) disposing the magnetically responsive particles into a flow chamber having a first and second port; (3) providing a source of a wash solution connected to the first port; (4) after (2), but prior, during, or after (3) immobilizing the magnetically responsive particles in a first magnetic field; (5) flowing the wash solution through the flow chamber; (6) optionally, arresting or attenuating the flow of the wash solution; (7) agitating the magnetically responsive particles by applying one or more complete or partial cycles of removing the first magnetic field, applying a second magnetic field, removing the second magnetic field, and applying the first magnetic field; and (8) optionally restoring flow of the wash solution.

In a preferred embodiment, the method further includes: (9) disconnecting the first port from the wash solution source; (10) providing a source of an elution solution connected to the second port; (11) flowing the elution solution through the flow chamber; and (12) collecting eluate from the first port.

The order need not be sequential. For example, (2) can be effected prior to (1). In particular, the magnetically responsive particles can be disposed in the flow chamber, e.g., in a buffer solution, and then contacted with a sample. In a preferred embodiment, the steps are performed in sequential order.

In a preferred embodiment, the method is such that the elution solution flows directly from the first port into a collection chamber, e.g., without contacting a fluid connector, e.g., a tubing or other adaptor. The collection chamber, e.g., tube or microtitre well, can be disposed directly beneath the first port. For example, an air gap can be provided between the first port and the collection chamber.

In another preferred embodiment, steps (5) to (8) (e.g., (5) and (7) or (5), (6), (7), and (8)) are repeated one or more times to wash the magnetically responsive particles.

In one embodiment, the elution solution includes a modifying or competing agent. The modifying agent can be a protease, e.g., a site-specific protease, e.g., thrombin or Factor VIII The protease can cleave members of the sample, e.g., to elute the nucleic acid component of bound display library members or cleave a protein attached to the magnetically responsive particle, e.g., thereby releasing a fragment or causing a conformational change (e.g., cleaving a viral surface protein such as hemagglutinin). The competing agent can be identical to a target attached to the magnetically responsive particles.

In another embodiment, the elution solution has a higher ionic strength than the wash solution. In still another embodiment, the elution solution has a lower ionic strength than the wash solution. In another related embodiment, the conditions of the elution solution alter the hydrophobic effect relative to the conditions of the wash solution.

In a preferred embodiment, the elution solution has a variable concentration of a component. For example, the elution solution can be flowed as a step or continuous gradient. The amount of salt or the pH can be varied across the gradient. In another preferred embodiment, at least part of the elution solution is collected in fractions, e.g., of fixed volume or corresponding to regular time intervals. When fractionated in combination, e.g., with elution using a competing agent, eluted components of the same can be separated relative to their dissociation constant for binding to the target.

In one embodiment, the wash solution includes a modifying or competing agent. In still another embodiment, the magnetically responsive particles are contacted to the sample in the presence of a modifying or competing agent, e.g., added to the sample before, after, or concurrent with the magnetically responsive particles.

The method can, of course, be practiced with an apparatus described herein and/or with any selection of features described herein.

In another aspect, the invention features a method of washing magnetically responsive particles. The method includes: (1) contacting magnetically responsive particles to a sample; (2) inserting the magnetically responsive particles into a flow chamber; (3) immobilizing the magnetically responsive particles in a first magnetic field; (4) flowing a liquid through the flow chamber in a first direction; (5) removing (or dissipating) the first magnetic field; and (6) immobilizing the magnetically responsive particles in a second magnetic field.

The method can further include: (7) removing the second magnetic field; (8) immobilizing the magnetically responsive particles in the first magnetic field; and (9) repeating steps (5) to (8) at least twice. The order need not be sequential. For example, (2) can be effected prior to (1). In a preferred embodiment, the steps are performed in sequential order.

In another aspect, the invention features a method of isolating a display library member. The method includes: providing an apparatus described herein to apply a method described herein, e.g., to thereby isolate a display library member.

In a preferred embodiment, the method includes: providing a flow chamber that includes a mixture comprising (a) magnetically responsive particles, (b) and members of a display library, the magnetically responsive particles having attached thereto and (c) a target compound that is attached or attachable to the magnetically responsive particles such that the target compound, particles, and at least some of the members of the display library form a complex; and washing at least some of the members from the particles by effecting one or more cycles that comprise (i) flowing a first solution through the flow chamber while the particles are captured by a magnetic field, and (ii) agitating the particles by selectively applying the magnetic field.

For example, the providing can include binding the target compound to a subset of the display library members, and, subsequently, attaching the target compound to the magnetically responsive particles. The flowing can remove a subset of display library members that do not bind to the target compound under conditions of the flowing.

In one embodiment, the method further includes isolating the subset of display library members that do not bind the target compound under conditions of the flowing.

In another embodiment, the method further includes: washing (e.g., “eluting”) at least some others of the members from the particles by effecting one or more cycles that comprise (iii) flowing a second solution through the flow chamber while the particles are captured by a magnetic field, and (iv) agitating the particles by selectively applying the magnetic field. The members that are washed from the particles can be collected and analyzed. The first and second solutions can have different ionic strengths, different pH's, or different agents. For example, the second solution can include a protease. In another example, the first solution maintains disulfide bonds and the second solution reduces disulfide bonds. In still another example, the second solution includes a competing agent, e.g., an agent identical to the target.

In one embodiment, the second solution has a variable concentration of a component, e.g., the second solution is supplied as a gradient such as a step or continuous gradient.

The first solution and/or second solution can be collected in fractions.

In one embodiment, the method includes amplifying at least some of the display library members in the second solution and repeating the method.

The first and/or second solution can include a medium for cell growth, e.g., a growth factor, a sugar, or other nutrients. The method can, of course, be practiced with an apparatus described herein and/or with any selection of features described herein.

In another preferred embodiment, the method includes: providing an apparatus described herein and magnetically responsive particles exposed to a sample; flowing a first solution in a first direction (e.g., vertically upwards) to wash the particles, then flowing a second solution in a second direction, opposite to the first (e.g., vertically downwards). In a preferred embodiment, the second solution is collected without contact to a fluid connector, e.g., tubing, e.g., plastic tubing. The method can, of course, be practiced with an apparatus described herein and/or with any selection of features described herein.

In another aspect, the invention features a method of refining a display library (e.g., removing members that bind a non-target at various stringencies). The method includes: combining magnetically responsive particles and members of a display library in a solution, the magnetically responsive particles having attached thereto a non-target compound; disposing the solution in a flow chamber; agitating the magnetically responsive particles by selectively applying a first and second magnetic field; flowing a fluid into a first port of the flow chamber; and collecting effluent from a second port of the flow chamber. The collected effluent can include members of the display library that are not bound to the non-target compound. The fluid can have equilibrium binding conditions that are more stringent than the equilibrium binding conditions of the solution in which the magnetically responsive particles and the display library are combined. In another embodiment, the fluid increases the dissociation rate of at least some library members for the non-target compound relative to the dissociation rate in the solution.

In one embodiment, the effluent is collected in fractions. In another embodiment, the initial effluent is, for example, discarded. The effluent is collected after an interval during which the fluid is flowed. In another embodiment, a subset of the members of the display library that competes with a compound in the solution are collected.

The methods described herein can be used, e.g., to separate a compound of interest from another compound or a mixture, to prepare or modify a compound, to separate cells, and to prepare magnetically responsive particles (e.g., pre-washing or modification).

In another aspect, the invention features a method that includes: forming a complex, in a vessel, that includes (a) magnetically responsive particles, (b) a target that is attached or attachable to the particles, and (c) a first replicable entity; applying a magnetic field that retains the complex in the vessel; removing fluid from the vessel; supplying a solution that supports replication to the vessel; and replicating the first replicable entity by one or more cycles of replication. The replication occurs in the presence of the target. The first replicable entity can be that is a nucleic acid, cell or virus particle. As appropriate, replication can include: (i) nucleic acid amplification, (ii) cell division or (iii) virus infection and release. A “virus particle” refers to a virus or a viral protein coated structure that can deliver a nucleic acid into a cell, e.g., a phagemid vector. The phagemid vector can be replicated in the host cell. Optionally, helper phage can be provided to produce more virus particles that encapsulate the phagemid vector. Virus particles include viruses and virus-like particles of viruses that infect mammalian cells, e.g., retroviruses.

In a related aspect, the invention features a method that includes: providing, in a vessel, that includes (a) magnetically responsive particles, to which a target is attached (or attachable) to the particles, and (b) replicable entities; binding a first subset of the replicable entities to the target; applying a magnetic field to capture the particles and the first subset of replicable entities; separating a second subset of the replicable entities from the first subset, the replicable entities of the second subset not binding to the target; and replicating the replicable entities of the first subset, e.g., under conditions for nucleic acid amplification, cell or virus growth. The replicable entities can be that are nucleic acids, cells or virus particles

The method can further include dissipating the magnetic field. For example, the magnetic field is applied by a magnetic field inducer positioned external to the vessel. In another example, the magnetic field inducer is applied by inserting a magnetic field inducer (e.g., a magnetic rod) into the vessel. The inserted magnetic field inducer can include a sheath.

In one embodiment, the separating comprises inserting the magnetic field inducer and captured first subset into a second vessel. The second vessel can include a growth medium. The separating can include flushing at least some of the fluid from the vessel while the entities of the first subset are captured.

The method can include repeated cycles of growth and solution replacement while bound members are captured by magnetically responsive particles. For example, recovered replicable entities can be isolated from solution removed from the vessel. In another example, the magnetically responsive particles are isolated, and the bound members are analyzed.

Examples of replicable entities include a nucleic acid (e.g., a nucleic acid-peptide fusion, a ribosome displayed polypeptide), a cell (e.g., a yeast cell displaying a heterologous polypeptide, an immune cell, another mammalian cell, a bacterial cell displaying a heterologous polypeptide), a virus (e.g., a bacteriophage displaying a heterologous polypeptide, or a modified mammalian virus).

In one embodiment, the vessel is a flow chamber. In another embodiment, the vessel has only a single port.

In another aspect, the invention features a method that includes: forming a complex, in a vessel, that includes (a) an insoluble support, (b) a target is attached or attachable to the insoluble support, and (c) a first replicable entity; and replicating the first replicable entity by one or more cycles of replication in the presence of the target. The first replicable entity can be that is a nucleic acid, cell or virus particle. As appropriate, replication can include: (i) nucleic acid amplification, (ii) cell division or (iii) virus infection and release. A “virus particle” refers to a virus or a viral protein coated structure that can deliver a nucleic acid into a cell, e.g., a phagemid vector. The phagemid vector can be replicated in the host cell. Optionally, helper phage can be provided to produce more virus particles that encapsulate the phagemid vector. Virus particles include viruses and virus-like particles of viruses that infect mammalian cells, e.g., retroviruses.

The separating can include flushing (e.g., flowing fluid across the insoluble support, or replacing fluid in contact with the insoluble support)at least some of the fluid from the vessel while the entities of the first subset are captured.

The method can include repeated cycles of growth and solution replacement while bound members are captured by the insoluble support. For example, recovered replicable entities can be isolated from a solution removed from the vessel. In another example, entities that are bound to the insoluble support are analyzed.

Examples of replicable entities include a nucleic acid (e.g., a nucleic acid-peptide fusion, a ribosome displayed polypeptide), a cell (e.g., a yeast cell displaying a heterologous polypeptide, an immune cell, another mammalian cell, a bacterial cell displaying a heterologous polypeptide), a virus (e.g., a bacteriophage displaying a heterologous polypeptide, or a modified mammalian virus).

In one embodiment, the vessel is a flow chamber. In another embodiment, the vessel has only a single port.

In one aspect, the invention features a method that includes:

a) forming a mixture comprising a plurality of diverse replicable entities, a target, and a support, wherein each replicable entity of the plurality displays a protein component that is heterologous to the replicable entity and each replicable entity includes a nucleic acid encoding the heterologous protein component, the heterologous protein component being a member of a set of diverse protein components;

b) forming replicable entity-immobilized target complexes, each of which comprises a replicable entity from the plurality which binds the target and the target immobilized to the support;

c) separating diverse replicable entities that do not bind to the target from the replicable entity-immobilized target complexes;

d) producing copies of the replicable entities in the presence of the target thereby forming copied entity-immobilized target complexes; and

e) separating copies of the replicable entities that do not bind to the target from the copy-entity-immobilized target complexes.

The method can be used to select replicable entities (e.g., display phage or display cells) that encode a target binding protein from a library.

In one embodiment wherein the replicable entity is bacteriophage, the step d) of producing can include contacting replicable entities from the phage-immobilized target complexes with host cells so that the host cells are infected by the replicable entities from the replicable entity-immobilized target complexes. In an embodiment wherein the replicable entities are phage, the method can further include one or more of the following features: fewer than 5000, 4000, 2000, 1000, 700, 500, or 300 progeny phage are produced for each phage that infects one of the host cells; the producing is completed in less than 4, 3, 2 1.5, 1, or 0.5 hours; the host cells divide less than seven, six, five, four or three times; and, an antibiotic whose resistance is encoded by a nucleic acid within each phage is present or absent. Time between the contacting d) and the separating e) can be less than 240, 120, 90, 80, 60, 45, 40, or 30 minutes and may be at least 30, 45, 60, 80, or 90 minutes.

In another aspect, the invention features a method that includes:

a) providing a library of replicable entities that each have a heterologous protein component that is physically attached to the respective replicable entity, wherein each protein component is a member of diverse set of different proteins;

b) contacting replicable entities of the library to a target;

c) performing one or more cycles of: i) forming replicable entity-immobilized target complexes, each of which includes (1) a replicable entity that binds to the target by its heterologous protein component and (2) the target immobilized to a support; ii) separating replicable entities that do not bind to the target from the replicable entity-immobilized target complexes, iii) producing copies of the replicable entities in the presence of the target, the produced copies being replicates of replicable entities that bind to the target; and

d) recovering the nucleic acid encoding the heterologous protein component of one or more produced replicable entities that bind to the target, thereby selecting a nucleic acid that encodes a binding protein for the target.

The method can be used to select a nucleic acid that encodes a binding protein from a library of display proteins. Examples of replicable entities include a nucleic acid (e.g., a nucleic acid-peptide fusion, a ribosome displayed polypeptide), a cell (e.g., a yeast cell displaying a heterologous polypeptide, an immune cell, another mammalian cell, a bacterial cell displaying a heterologous polypeptide), a virus (e.g., a bacteriophage displaying a heterologous polypeptide, or a modified mammalian virus).

In one embodiment wherein the replicable entity is bacteriophage, the step iii) of producing can include contacting replicable entities from the phage-immobilized target complexes with host cells so that the host cells are infected by the replicable entities from the replicable entity-immobilized target complexes. In one embodiment wherein the replicable entity is bacteriophage, the step of producing can include contacting replicable entities from the phage-immobilized target complexes with host cells so that the host cells are infected by the replicable entities from the replicable entity-immobilized target complexes. In an embodiment wherein the replicable entities are phage, the method can further include one or more of the following features: fewer than 5000, 4000, 2000, 1000, 700, 500, or 300 progeny phage are produced for each phage that infects one of the host cells; the producing is completed in less than 4, 3, 2 1.5, 1, or 0.5 hours; the host cells divide less than seven, six, five, four or three times; and, an antibiotic whose resistance is encoded by a nucleic acid within each phage is present or absent. Time between the contacting and the separating can be less than 240, 120, 90, 80, 60, 45, 40, or 30 minutes and may be at least 30, 45, 60, 80, or 90 minutes.

In another aspect, the invention features a method of amplifying a plurality of display library members. The method includes: amplifying a plurality of display library members in the presence of a target (e.g., a target compound or target cell) to yield a population of amplified display library members (e.g., bacteriophage display library members or cell display library members). In one embodiment, during or after the amplifying, at least a subset of the amplified display library members physically interact with the target compound. In one embodiment, at least a subset of the amplified display library members bind to the target compound. The method can further include a subset of the amplified display library members that bind to the target compound. The method can include other features described herein.

In another aspect, the invention features an apparatus that includes: (a) a vessel that includes (1) a growth medium, (2) cells that are display library members or cells injectable by such (e.g., a bacteriophage display library member), and (3) magnetically responsive particles having a target compound attached thereto (or attachable thereto); and (b) a magnetic field inducer configured to capture the particles as the growth medium in the vessel is replenished.

In still another aspect, the invention features an apparatus that includes: (a) a vessel that includes (1) reagents for DNA synthesis, (2) entities that are nucleic acid library members, and (3) magnetically responsive particles having a target compound attached thereto (or attachable thereto); and (b) a magnetic field inducer configured to capture the particles as the ingredients required for DNA synthesis in the vessel is replenished. The entities can encode, e.g., peptide or polypeptides. The peptide or protein is, for example, attached or attachable to the nucleic acid that encodes it (e.g., by way of a covalent or non-covalent attachment, or by encapsulation, e.g., by a cell membrane or viral wall. The vessel can further included reagents required for protein synthesis.

In still another aspect, the invention features a kit that includes magnetic beads and a capillary tube. The kit can also include an apparatus described herein, tubing (adapted to fit an apparatus or capillary tube described herein), and/or a display library. The magnetic beads can have a surface that includes a reagent that binds to a peptide tag. For example, the reagent can be an immobilized metal (e.g., nickel-nitrilotriacetic acid (NTA-Ni²⁺) and other NTA-bound metals), an antibody that recognizes a specific epitope, or a ligand for a polypeptide such as glutathione, biotin, chitin, and maltose.

As used herein, a “polypeptide” is a polymer of amino acids of any length greater than three, e.g., inclusive of a peptide or protein. A “short peptide” is a polypeptide of less than 30 amino acids. A “protein” can include one or more polypeptide subunits.

As used herein, a “display entity” is an entity that includes an accessible polypeptide component and a recoverable nucleic acid component that encodes or identifies the polypeptide component. In one embodiment, the display molecule is attached to the nucleic acid component. For example, the polypeptide component is attached to a viral coat that encapsulates the nucleic acid component. In another example, the polypeptide component is covalently linked to the nucleic acid component. In still another embodiment, the display molecule is not attached to the nucleic acid component, but is attached to a tag (e.g., a tag which can itself be a polypeptide or nucleic acid sequence or a non-biological tag) that includes sufficient information in the context of its application to identify a nucleic acid sequence that encodes the polypeptide component. For example, the tag can be a radiofrequency transponder that includes an identifier associated with a data string indicating the polypeptide sequence of the polypeptide component. The nucleic acid component is “recoverable” as its sequence can be inferred from the data string and from which it can be synthesized.

A “display library” is a collection of at least three display entities. The nucleic acid components of the library (and hence, the polypeptide components) can include a segment (e.g., at least a nucleotide) that is diversified. Segment diversity can be random, partially random, designed, or natural. An example of a random segment is a segment synthesized from degenerate oligonucleotides. An example of a partially random library is library whose members include a segment synthesized from a partially random pool of oligonucleotides, e.g., using biased nucleotide pools, or selected or biased trinucleotide units. An example of a designed library is a library formed from particular known individual members, e.g., a subset of arrayed individual members of a random library or members designed by a computer program. An example of a library that includes natural diversity is a library constructed from naturally occurring sequence segments (e.g., sequences encoding naive immunoglobulin variable domains). A variety of formats for display libraries are known in the art, see, e.g., Li et al. (2000) Nat. Biotechnol. 18:1251-1255. Some preferred display formats are described herein. One preferred display format is phage display. One other preferred format is yeast display.

A “magnetically responsive particle” is a magnetic or paramagnetic particle which is magnetized or magnetizable to the extent that the particle moves in a magnetic field, if present. The particle can be magnetizable only transiently, e.g., only when a magnetic field is present. The particle can be any convenient shape, e.g., spherical, oblong, flat, and so forth.

As used herein, a “capillary” or “capillary flow chamber” refers a chamber whose an internal diameter is less than 4 mm. An example of a capillary is a glass capillary with an internal diameter of less than 1.8 mm.

The details of one or more embodiments of the invention are set forth in the accompanying drawings and the description below. Other features, objects, and advantages of the invention will be apparent from the description and drawings, and from the claims. All patents and references cited herein, including U.S. Provisional Patent Application Serial No. 60/337,775, filed Dec. 7, 2001, and No. 60/408,624, filed Sep. 5, 2002, are incorporated in their entireties by reference for all purposes.

### BRIEF DESCRIPTION OF THE DRAWINGS

FIGS. 1 to 3 are schematics of an exemplary apparatus that includes a flow chamber and two magnetic field inducers.

FIGS. 4 to 8 are diagrams of an exemplary apparatus.

FIG. 9 is a schematic of an exemplary system.

FIG. 10 is a schematic of an exemplary fermentor.

### DETAILED DESCRIPTION

The invention provides an apparatus and methods for washing magnetically responsive particles. Generally, magnetically responsive particles having a particular surface property are exposed to a sample of biological molecules. The particles are then washed using the method and/or an apparatus described herein.

First, one exemplary magnetically responsive particle washing apparatus is described.

Washing Apparatus

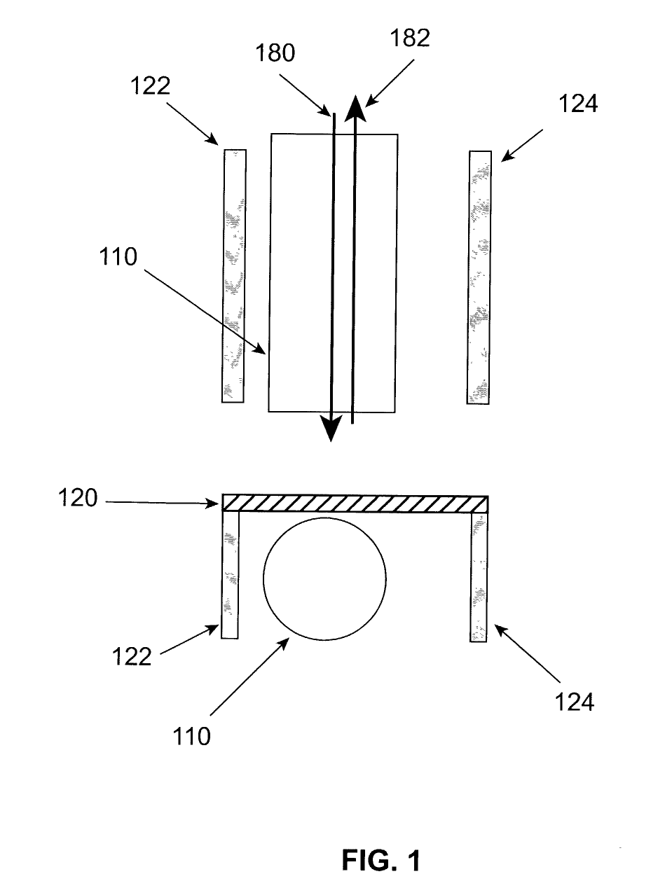

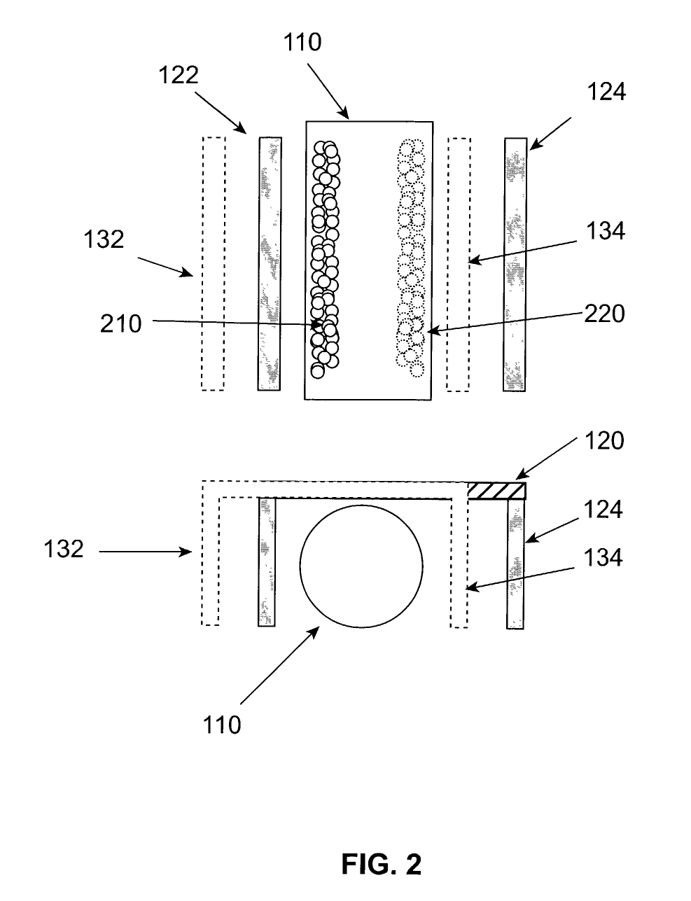

Referring to the example depicted in FIGS. 1 and 2, a magnetic bead washing apparatus includes a chamber 110 for housing magnetic beads 210. Liquid can flow through the chamber 110 along the path 180 or the path 182. The chamber 100 can be, for example, a glass capillary tube of diameter approximately 1.4 mm and length approximately 140 mm.

The chamber 110 is located between a first magnet 122 and a second magnet 124. The magnets 122 and 124 are attached to a frame 120. The frame can be actuated from a first position to a second position. When the frame is actuated, the first magnet 122 is actuated to a second position 132 and the second magnet 124 is actuated to a second position 134.

In the first position 126, the first magnet 122 produces a magnetic field in a first zone 210 of the chamber 110. In the second position 136, the second magnet 124 produces a magnetic field in a second zone 220 of the chamber 110. The actuation of the frame also removes the magnetic field from the first zone 210. In this example, the first and second zones are diametrically opposed. The alteration of magnetic field results in movement of magnetic beads from the first zone 210 to the second zone 220.

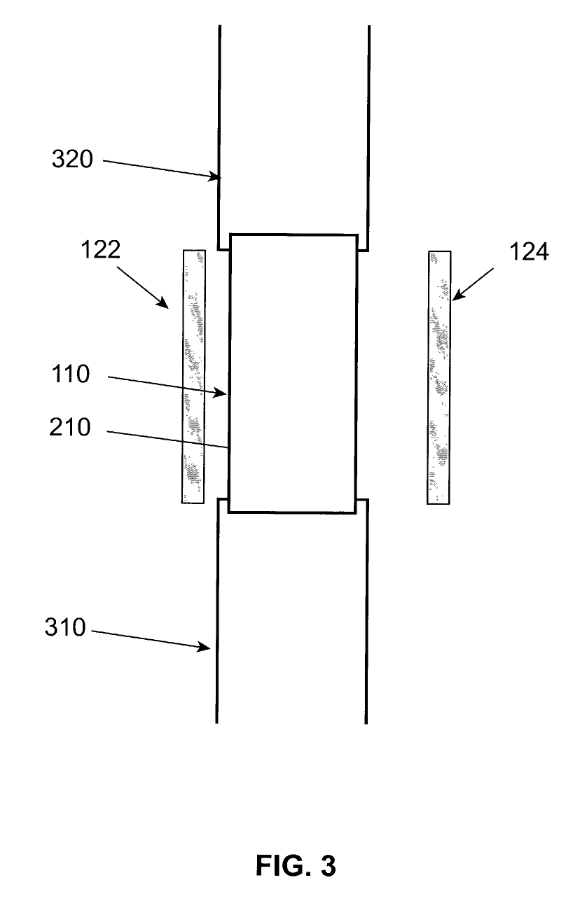

Referring also to FIG. 3, the chamber 110 can be attached to fluid connectors 310 and 320. During a wash step, the chamber 110 can be attached to both fluid connectors 310 and 320. Fluid can enter the chamber from the bottom connector 310 and leave via the top connector 320. The wash is in the upwards direction 180.

During an elution step, the chamber 110 may only be attached to the top fluid connector 320. Fluid, e.g., the elution solution, enters the chamber from the top, and leaves at the bottom. The elution solution can be dispensed directly into a collection vessel, e.g., without contacting a connector such as a tubing, frit, or other adaptor.

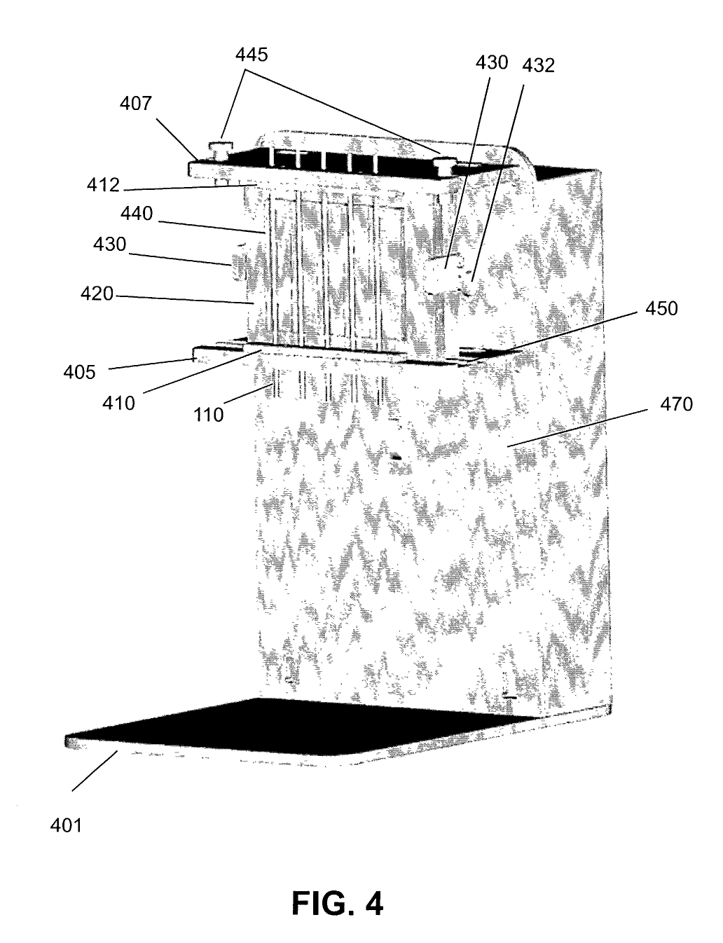

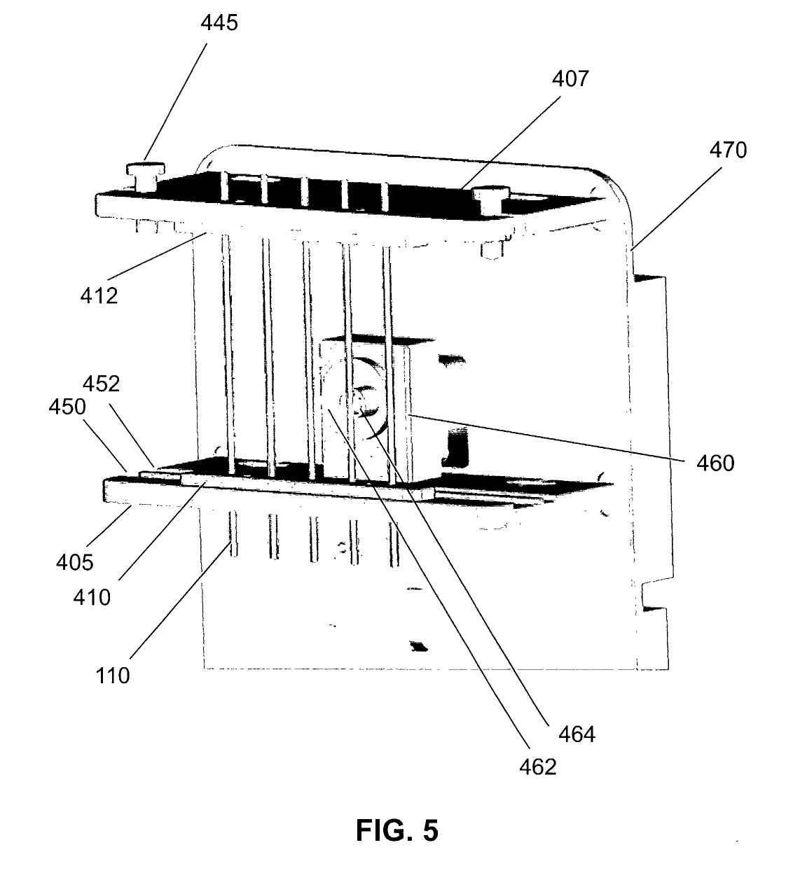

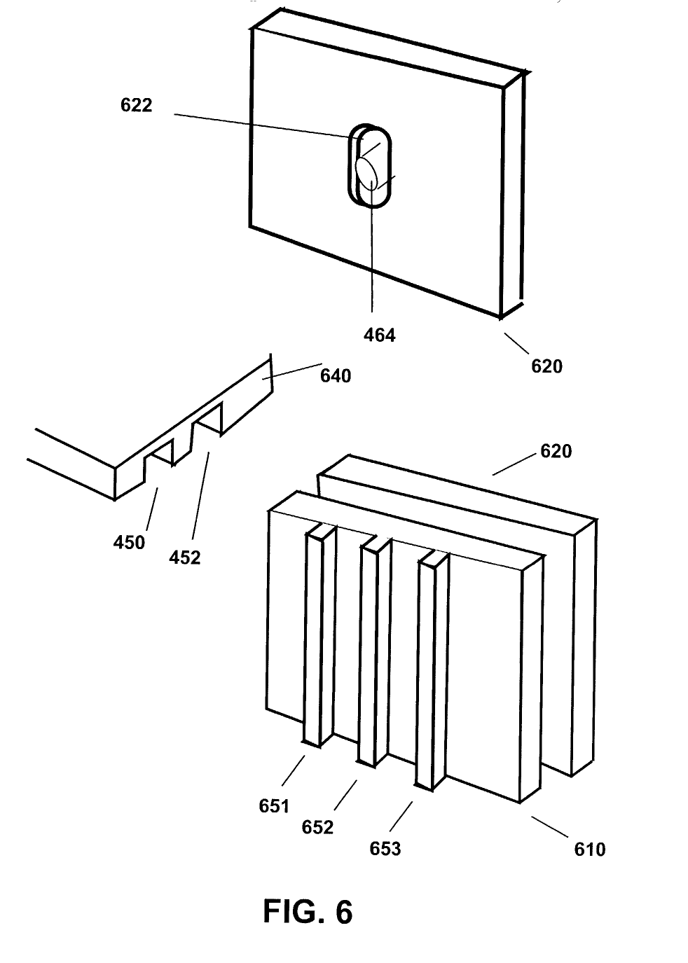

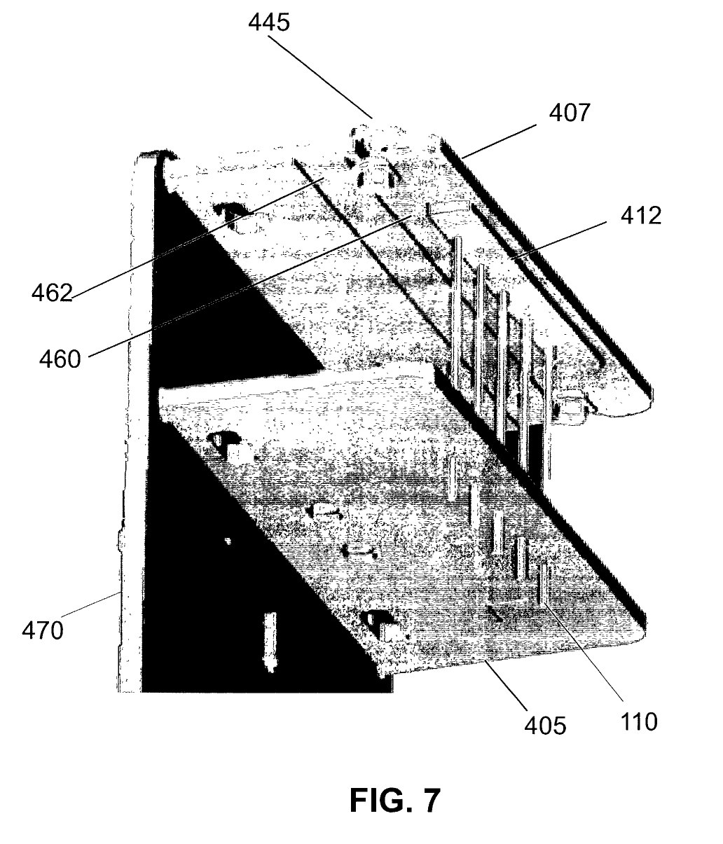

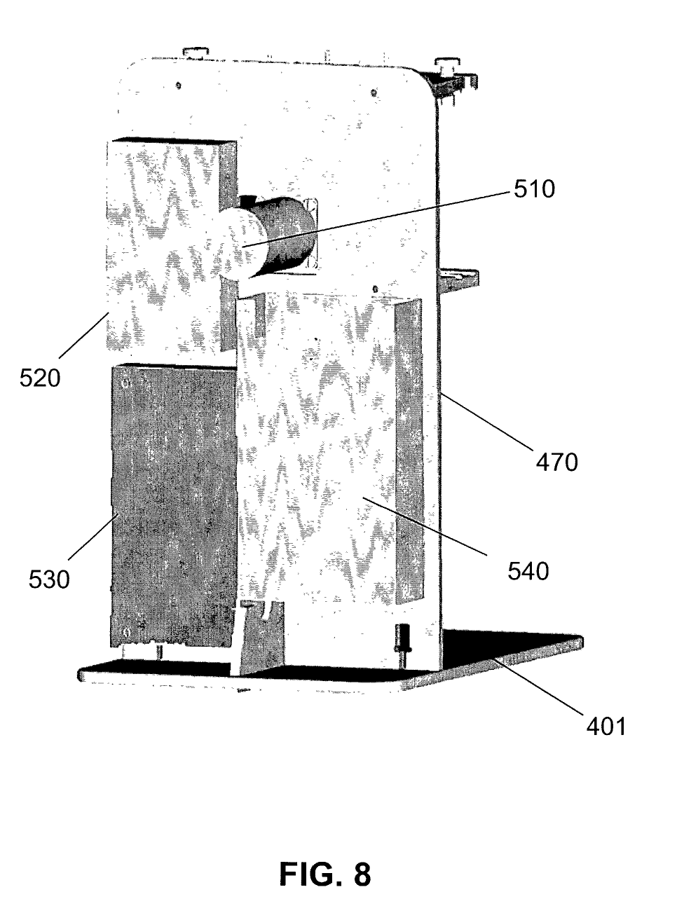

Referring to the example in FIGS. 4 to 8, an apparatus 400 includes a horizontal base 401 and a vertical stand 470. The stand 470 includes a lower shelf 405 and an upper shelf 407. The shelves 405 and 407 are supports for the capillary flow chamber.

The shelves include fittings for holding capillary tubes 110. These fittings are bores that are reinforced by brackets 410 and 412. Other fittings can include clamps, clasps, and so forth.

The shelves 405 and 407 include two parallel recessed tracks 450 and 452 in which the frame 420 can slide. The frame can include a front plate 610 and a rear plate 620. The rear plate 620 is secured to the front plate 610 by brackets 430 which can be tightened by knobs 432. The two plates 610 and 620 are positioned to slide in parallel in the recessed tracks 450 and 452. Removable screws 445 in the top shelf 407 retain the plates on the tracks.

The front plate 610 supports the base 440, to which a plurality of magnetic field inducers is attached. In this example, the magnetic field inducers are magnets (e.g., 651, 652, and 653) spaced at regular intervals.

The rear plate 620 can include an oval aperture 622. The spindle 464 of a rotating cam 462 is located in the aperture 622. When the cam rotates, the spindle 464 is eccentrically driven. The rear plate 620, and thereby the frame 420, is reciprocated along the tracks 450 and 452 when the cam rotates.

Flow Chambers. Flow chambers can be designed for any capacity, e.g., by judicious design of the length and cross-sectional area of the chamber. In one embodiment, the flow chamber has a cross-sectional area of less 4 mm². In such narrow flow chambers that are vertically positioned, magnetically responsive particles can be displaced from one zone of the chamber to another zone without the particles settling to lower regions of the chamber.

The inner surface of non-glass flow chambers can be coated, e.g., silanized, or otherwise modified to reduce binding to biological compounds such as phage, cells, proteins, and nucleic acids.

One exemplary flow chamber is a glass tube. For example, the glass tube can have an internal diameter of between 0.4 to 1.3 mm, e.g., 1.4 mm 10.05 mm and a length of about 140 mm. The volume of such a tube is about 200 μl. The tube can have minimal taper, e.g., a taper of less than 0.02 mm. The tube can be formed of borosilicate glass. Some such tubes are commercially available as glass capillary tube (e.g., as sold for blood drawing).

The inner surface of a flow chamber can be smooth or rough. A roughened inner surface can increase the exposed surface area on paramagnetic particles when they are captured against the inner wall of the flow chamber.

The use of a narrow flow chamber with a small volume can provide a number of advantages. For example, as a result of the small volume, at reasonable flow rates, a newly applied solution contacts particles at the top of the flow chamber at close to the same time as particles at the bottom of the flow chamber. On the other hand, because the chamber is narrow, in gradients of solutions can be applied without undue mixing between layers.

Additional Features of the Apparatus

The apparatus 400 can include various additional features. Non-limiting examples include the following:

Spectrophotometer. The effluent line can be fitted with an optical flow cell, e.g., the Amersham-Pharmacia UW-11 model. The optical flow cell includes a light source, e.g., of wavelengths of 260 nm, 280 nm, or 340 nm. The optical flow cell also includes a detector that measures the amount of light absorbed by fluid in the flow cell. Output from the detector can be monitored by a chart recorder or by a computer that is interfaced with the detector and that stores information from the detector. A user can monitor the chart recorder or the computer to determine the amount of UV-absorbing material leaving the flow chamber 110. Since both proteins and nucleic acid absorb UV-light, the user has an indication of the extent of protein and nucleic acid leaving the flow chamber 110. Such information can be used to manually or automatically determine the required length of washes or elution procedures. In the automatic mode, the computer is programmed to detect threshold absorbance values and trigger events accordingly. For example, the computer can switch a fluid driver from delivering a wash solution to an elution solution 20 minutes after the effluent line attains and maintains a UV-absorbance equivalent to the wash solution.

In some embodiments, the optical flow cell monitors the scattering of visible light or fluorescence. The density of cells in solution, for example, is proportional to the amount of light scattered by the solution. In implementations in which cells and viruses are fluorescently detectable (e.g., labeled), internally or externally, the number of cells or viruses leaving the flow chamber 110 can be measured by monitoring fluorescence in the optical flow cell.

Cells can be fluorescently detectable if they express a fluorescent protein, e.g., green fluorescent protein or a variant thereof. Cells can also be fluorescently labeled by modification of cell surface proteins with a fluorescent dye, e.g., Cy3. For example, cell surface proteins can be biotinylated then bound with Cy3-labeled avidin. Further, cells can be loaded with a fluorescent dye that is membrane permeant and once and esterified is maintained as a stable, cytoplasmic fluorophore (e.g. calcien-AM, available from Molecular Probes, Eugene Oreg.). Additionally cells can be labeled with lipophilic fluorophores either directly (e.g. DiI, available from Molecular Probes) or metabolically using pyrene labeled fatty acids (e.g. 1-pyrene decanoic acid, available from Molecular Probes) that are incorporated into the lipid bilayer.

Viruses can be labeled, e.g., by chemically coupling a fluorescent dye to an external viral coat protein or by incorporated a fluorescent protein into the viral particle. For example, a fluorescently labeled antibody can be bound to a virus coat protein, e.g., to a major or minor coat protein, preferably a coat protein not required for viral infectivity, e.g., a coat protein other than Gene III.

Sensor in the Effluent Line. The apparatus can include additional sensors in the effluent line. These can be used to monitor other parameters of the effluent material. For example, a conductivity meter can be used to monitor the ionic strength of the effluent. Such information is particularly useful to generate associations between samples of the effluent and the concentration of buffers during a gradient elution. As described above, such information can be communicated to a computer or displayed by a chart recorder. Similarly, a pH sensor can be used. If the volume of the flow chamber 110 is small (e.g., the flow chamber 110 is a capillary tube), a pH sensor and conductivity meter can also be fitted in the input line.

Fraction Collector. To collect material that emerges from the flow chamber 110, whether during a wash or an elution phase, samples can be collected manually or automatically. In a preferred embodiment, the sample is collected directly from one of the ports of the flow chamber. For example, a tube can be positioned directly underneath a capillary tube flow chamber. In this configuration, the effluent does not contact a plastic or polymeric tubing which might non-specifically absorb biomolecules from the effluent or which might release previously-absorbed biomolecules. For implementations for which low-level non-specific absorption is not consequential or is inadvertent, tubing, e.g., a disposable tubing, can be used to connect fluid flow form the flow chamber to the fraction collector or more particularly to a vessel manipulated by the fraction collector.

Sensor in the Input Line. A variety of sensors can be positioned in the input line. For example, a pressure sensor can be used to monitor backpressure in front of the flow chamber 110. The pressure sensor can be configured to stop the fluid driver. Another useful sensor is an air detector that detects air in the input line. Air can enter the line, for example, if a fluid reservoir is emptied, or if a fault arises in a fitting or joint in the fluid connection.

Fluid Driver. Examples of fluid drivers include a peristaltic pump and a two-chambered piston-driven pump, e.g., Amersham-Pharmacia P-500 or P-903. The pumps are electronically controlled and can be regulated to deliver fluid under positive pressure at very precise flow rates. For example, the P-903 has provides a continuous flow at rates of 0.001 to 10 ml/minute with only a 2% deviation.

Peristaltic pumps can be used to drive fluid under positive pressure into the flow chamber. The peristaltic pump can also be positioned in the effluent line in order to draw fluid from the flow chamber, e.g., using negative pressure. This configuration can be used, e.g., during the wash phase. One exemplary peristaltic pump is the Watson-Marlow Type 20548-channel peristaltic pump. This pump can be electronically interfaced to a controller and operated using software-generated instructions. The Bio-Rad EP-1 Econo pump (Bio-Rad Laboratories, Hercules Calif.) is another exemplary peristaltic pump that can provide a controlled flow rate of 0.01 to 20 ml/minute. Tubing with internal diameters of between 0.8 and 3.2 mm can be fitted onto the pump. An appropriate size tubing is chosen depending on the desired flow rate.

The apparatus 400 can also include reservoirs for holding buffers, e.g. a buffer A and buffer B, which can be combined in various proportions. For example, the Amersham-Pharmacia Gradient Programmer GP-250 Plus and two Amersham-Pharmacia High Precision P-500 pumps can be configured to provide a gradient of buffer A and B to the flow chamber. Buffer A can be a low salt buffer, e.g., 50 mM Tris HCl, 50 mM KCl pH 7.5. Buffer B can be a high salt buffer, e.g., 50 mM Tris HCl, 800 mM KCl pH 7.5. The buffers can be mixed in a step, linear, or non-linear gradient. See, e.g., Scopes (1994) Protein Purification: Principles and Practice, New York: Springer-Verlag.

Magnetic Field or Magnet Position Sensor. The apparatus can also include sensors that detect the presence of a magnetic field or that detect the position of a magnet (e.g., directly or indirectly by determining the location of the magnet or an indicator attached to the magnet, such as the frame). The sensors can send information to a processor controlling displacement of the magnet or activation the magnetic field. Such information can be used to verify that commands have been correctly executed by the apparatus.

Temperature Sensor. The apparatus can include a temperature sensor to determine or estimate the temperature of the fluid in the flow chamber. For example, if the flow chamber is jacketed, the sensor can measure the temperature of the jacket. If the flow chamber is in a refrigerated housing, the sensor can measure the air temperature in the housing. In one embodiment, the sensor directly measures the temperature of fluid in the flow chamber, fluid entering the flow chamber, or fluid exiting the flow chamber.

Other Embodiments of the Apparatus

A number of other embodiments of the apparatus can be used. One exemplary embodiment includes a cartridge that includes a plurality of channels, e.g., four channels. Each channel functions as a flow chamber that can be loaded with paramagnetic particles. The cartridge snaps into a housing that supports the cartridge and that delivers fluid. The housing, positions the cartridge, and thus all channels, between first and second magnetic field inducers. The cartridges can be configured to be disposable and/or to be provided pre-loaded with paramagnetic particles.

In another exemplary embodiment, a flow chamber is positioned horizontally. A single magnetic field is applied to the top of the chamber to immobilize paramagnetic particles against the top inner wall of the chamber. Fluid can be flowed through the chamber. To agitate the particles, the flow is arrested and the magnetic field is removed. The particles are allowed to settle to the lower inner wall of the chamber. The magnetic field is then reapplied. In this configuration, time is provided to allow the particles to adequately settle to the lower inner wall of the chamber. Although the method may require additional time relative to methods with two magnetic field inducers, it enables particularly gentle agitation of the particles as may be required by some materials, e.g., living cells.

In still another exemplary embodiment, multiple flow chambers are attached to a carousel. The carousel rotates chambers between different positions, e.g., positions for (1) mounting a new chamber, (2) loading a chamber with magnetic particles that have been contacted to a sample, (3) washing a chamber with a solution to remove non-specifically or weakly bound molecules, (4) eluting molecules from a chamber; and (5) disposing of a used chamber. Positions 3 and 4 can position a chamber between a first and second magnetic field inducer in order to agitate the magnetic particles during the washing steps.

Yet another embodiment, a fermentor for continuous growth and selection, is described below.

The apparati and methods herein can be adapted for high-throughput application, e.g., applications that isolate molecules using multiple chambers operating in tandem. The high throughput applications can include diagnostics (e.g., analyzing samples from multiple patients), genomics (e.g., isolating nucleic acids for gene mapping and gene sequencing projects), and proteomics (e.g., performing library against library screening to identify ligands for multiple polypeptides in the proteome).

Magnetically Responsive Particles

Magnetically responsive particles, e.g., as used herein, can include an attached target molecule or a binding agent that is used to probe or isolate one or more biomolecules from a solution.

Magnetically responsive particles include paramagnetic particles or beads. Examples of paramagnetic beads are described in U.S. Pat. No. 4,554,088 (paramagnetic bead with a metal oxide core and polymeric silane coat); U.S. Pat. No. 5,356,713 (magnetizable microsphere with a core of magnetizable particles and a hydrophobic vinylaromatic monomer casing); U.S. Pat. No. 5,395,688 (particle with a polymer core coated with a mixed paramagnetic metal oxide-polymer layer); and U.S. Pat. No. 4,774,265 (paramagnetic bead with a polymer core adsorbed with metal oxide).

Another exemplary magnetically responsive particle is the Dynabead®) available from Dynal Biotech (Oslo, Norway). Dynabeads® provide a spherical surface of uniform size, e.g., 2 μm, 4.5 μm, and 5.0 μm diameter. The beads include gamma Fe₂O₃ and Fe₃O₄ as magnetic material. The beads are superparamagnetic as they have magnetic properties in a magnetic field, but lack residual magnetism outside the field. The beads are available with a variety of surfaces, e.g., hydrophilic with a carboxylated surface and hydrophobic with a tosyl-activated surface. The beads can have a specific gravity in the range of 1.1 to 1.8, e.g., 1.2 to 1.5. Beads can be prepared as described in U.S. Pat. Nos. 4,336,173; 4,459,378; and 4,654,267.

Appropriate beads can be chosen according to the charge and hydrophobicity of the agents bound to the beads. The zetapotential of the bead surface can be measured to determine how a bead may electrostatically interact with an intended agent. Beads can also be blocked with a blocking agent, such as BSA or casein.

The target is attached to the paramagnetic particle directly or indirectly. A variety of target molecules can be purchased in a form linked to paramagnetic beads. In one example, a target is chemically coupled to a bead that includes a reactive group, e.g., a crosslinker (e.g., N-hydroxy-succinimidyl ester) or a thiol.

In another example, the target is linked to the bead using a member of a specific binding pair. For example, the target can be coupled to biotin. The target is then bound to paramagnetic particles that are coated with streptavidin (e.g., M-270 and M-280 Streptavidin Dynabeads® available from Dynal Biotech, Oslo, Norway). In one embodiment, the target is contacted to the sample prior to attachment of the target to the paramagnetic particles. Other specific binding pairs that include a small organic molecule and a polypeptide that specifically binds the molecule are listed in Table 1.

TABLE 1

| Protein | Ligand |
|---|---|
| glutathione-S-transferase, | glutathione |
| chitin binding protein | chitin |
| Cellulase (CBD) | cellulose |
| maltose binding protein | amylose, or maltose |
| dihydrofolate reductases | methotrexate |
| FKBP | FK506 |

Another class of specific binding pair is a peptide epitope and the monoclonal antibody specific for it (see, e.g., Kolodziej and Young (1991) Methods Enz. 194:508-519 for general methods of providing an epitope tag). Exemplary epitope tags include HA (influenza hemagglutinin; Wilson et al. (1984) Cell 37:767), myc (e.g., Mycl-9E10, Evan et al. (1985) Mol. Cell. Biol. 5:3610-3616), VSV-G, FLAG, and 6-histidine (see, e.g., German Patent No. DE 19507 166).

Another exemplary specific binding pairs includes a cell surface protein and an antibody that binds to it. The cell surface protein can be specific to a particular cell type or to a cell having a particular property, behavior or disorder. For example, the cell can be a cancer cell, and the antibody can bind specifically to hypoglycosylated MUC1, melanoma differentiation antigen gp100, or CEA.

Washing Magnetically Responsive Particles

The apparati described herein are designed for the controlled washing of magnetically responsive particles. The term “washing” is used in the most general sense, i.e., the application of a liquid flow. For example, the term is not limited to the concept of a buffer “wash” during ion exchange chromatography. Particles can be washed with a solution that includes a sample in order to deliver the sample to the particles, with a solution that removes molecules that are non-specifically or weakly bound to the particles, or with a solution that removes molecules that are specifically or tightly bound to the particles.

In the context of the apparatus depicted in FIG. 4, washing can be performed in accordance with the following exemplary method: A magnetic field is applied to the first zone of a capillary tube that is the flow chamber. The particles adhere to sidewalls of the capillary tube in the first zone. A flow of buffer is applied to the capillary tube. The magnetic field keeps the paramagnetic particles immobilized during liquid flow.

To displace the beads, the liquid flow is arrested. The magnetic field is removed from the first zone and a magnetic field is applied to second zone. Due to the narrow diameter of the capillary tube 110, the paramagnetic particles move horizontally from the first zone to the second zone without settling to the bottom of the flow chamber. The magnetic field applied to the second zone can maintain the beads against the sidewall of the second zone. Further during some procedures, the liquid flow is in an upwards direction. This directionality further minimizes settling of the paramagnetic particles to the bottom of the flow chamber or out of the flow chamber.

The displacement process can be repeated any number of times. Liquid flow can resume after the one or more displacement steps. Agitation caused by the displacement step increases the speed and efficiency of washing the beads. For example, agitation reorients the particles relative to the flow chamber interior wall, relative to each other, and relative to the direction of liquid flow. Agitation can dislodge debris or insoluble material that may have entered the flow chamber or that may have been produced in the flow chamber, e.g., due to aggregation, cell lysis and so forth. Further, the process of agitation itself can produce shear forces that remove bound, e.g., non-specifically bound, molecules from the particle.

The apparatus can also be used to pre-treat (e.g., rinse or modify), pre-elute, and/or equilibrate paramagnetic particles prior to contacting the particles to a sample.

The method can be further adapted to deliver different liquids. One exemplary adapted method includes segments for (1) loading of the paramagnetic particles; (2) flowing a solution to remove molecules not of interest; and (3) flowing an elution solution to remove molecule of interest. These segments are described as follows:

Loading paramagnetic particles in a capillary flow chamber. In one embodiment, paramagnetic particles are mixed with a sample in a tube, e.g., an Eppendorf™ tube. The particles can be incubated with the sample in the tube, e.g., with gentle rotation, until equilibrium binding conditions are attained. The particles can be washed in the tube prior to disposal in the capillary flow chamber

The binding conditions can be tailored to a desired stringency or specificity. Generally, to isolate biomolecules that bind a target with a minimal affinity, the amount of target attached to the paramagnetic particles is maintained at a concentration less than the desired affinity constant. Specificity can be imparted by including a competing molecule that is similar, yet distinct from the target. The competing molecule can be in excess of the target by about at least 10, 100, or 1000 fold. The presence of the competing molecule favors isolation of biomolecules that bind the target to a greater extent than the competing molecule.

Then, the particles are disposed in the capillary flow chamber, e.g., by pumping, positive pressure, or capillary action. The particles can be added in a volume that is smaller than the volume of the flow chamber. Preferably, the particles drawn into the capillary flow chamber directly, e.g., to avoid contact between a tubing or fitting and the sample (e.g., members of a display library). The capillary flow chamber can be mounted in the apparatus depicted in FIG. 4, before, during, or after the loading. After the particles are loaded, a first magnetic field is applied to immobilize them.

In another embodiment, the paramagnetic particles are loaded into the capillary while suspended in a buffer solution free of the sample. The particles are immobilized in the capillary using the first magnetic field. Then the sample is flowed through the capillary. The particles and sample can be incubated together in the capillary for a controlled length of time. For example, the incubation can be very brief by flowing the sample through rapidly. A brief incubation may enhance selection for biomolecule in the sample that have a rapid association rate (Kn) for the target attached to the particles. In another example, the incubation is extended. The sample is flowed into the capillary and then flow is arrested. The particles can be periodically agitated in the capillary during the incubation period.

Washing non-specifically or weakly bound biomolecules. Subsequent to an incubation of any duration or subsequent to loading a capillary tube with paramagnetic particles that were contacted to a sample, a rinsing solution is flowed through the flow chamber in order to remove non-specifically or weakly bound biomolecules.

A variety of conditions can be used to remove non-specifically or weakly bound biomolecules from the paramagnetic particles. These conditions can be judiciously chosen according to the intended application. A first wash solution can be used that is similar or identical to the buffer solution used for binding the sample of biomolecules to the particles.

Later wash solutions can be of increasing stringency. For example, the ionic strength of a wash solution can be increased, e.g., by increasing NaCl or KCl concentration. In another embodiment, the hydrophobic effect can be modulated, e.g., by varying the concentration of ammonium sulfate.

Elution. Elution solutions are flowed through the chamber to remove biomolecules that are bound to the target on the paramagnetic particles. An elution solution generally has a different property from a washing solution. For example, the elution solution can have a different temperature, pH, ionic strength, or concentration of a solute. For example, the elution solution can be acidic or basic relative to the wash solution. The elution solution can also have an increased concentration of divalent cations, chelating agents, reducing or oxidizing reagents, detergents, and chaotropes.

Specific solutes that compete with the target for the biomolecules bound to the target can be used to selectively elute biomolecules of interest. See also “Off-rate Selection” below.

In another embodiment, elution is effected by separating the target molecule from the paramagnetic particles. For example, the target molecule might be cleaved from the paramagnetic particles if it is attached by a linker peptide that includes a specific protease site. In another example, if the target molecule includes a hexa-histidine tag bound to a metal immobilized on the paramagnetic particle, the target molecule can be released from the particle by addition of imidazole, a metal chelator, or a reducing agent.

In one embodiment, an enzyme is used to modify the target attached to the paramagnetic particles or the collection of biomolecules bound to the target. For example, a protease can be flowed through the chamber at a particular concentration and flow rate. The protease can cleave biomolecules that include a specific site recognized by the protease. The cleaved biomolecules are then recovered. If the biomolecules are in the format of a display library, their sequence can be inferred. If the biomolecules are polypeptides, e.g., free polypeptides, mass spectroscopy can also be used obtain information about the cleaved polypeptides. Such information can be used to infer the cleavage site.

Enzymes, e.g., proteases, an enzyme that can remove a display library member from a support (see, e.g., U.S. Pat. No. 5,432,018).

Off-Rate Selection

Since a slow dissociation rate can be predictive of high affinity, particularly with respect to interactions between polypeptides and their targets, methods can be used to isolate biomolecules with a selected kinetic dissociation rate for a binding interaction to a target immobilized on paramagnetic particles.

Using an apparatus described herein, the paramagnetic particles are first washed with a first solution that removes non-specifically or weakly bound biomolecules. Agitation steps are include during the wash with the first solution.

Then the particles are washed (i.e., eluted) with a second solution that includes a saturation amount of free target, i.e., replicates of the target that are not attached to the particle. The free target binds to biomolecules that dissociate from the target molecules that is attached to the paramagnetic particles. Rebinding is effectively prevented by the saturating amount of free target relative to the much lower concentration of target attached to the particles.

The second solution can have solution conditions that are substantially physiological or that are stringent. Typically, the solution conditions of the second solution are identical to the solution conditions of the first solution. Agitations steps can also be included during the elution, i.e., the wash with the second solution.

The effluent from the flow chamber during the elution is collected in fractions, e.g., using a fraction collector or manually. Fractions are numbered in temporal order to distinguish early from late fractions. Later fractions include biomolecules that dissociate at a slower rate from the target than biomolecules in the early fractions.

The use of a capillary flow chamber having a small volume relative to the flow rate further facilitates the separation of biomolecules having differing dissociation rates since the biomolecules leave the chamber into a collection tube rapidly after dissociating. Frequent and rapid agitation steps can be applied during the elution in order to minimize the non-specific trapping of eluted materials. Such steps improve the ability to separate biomolecules with respect to dissociation rate.

In one embodiment, the flow rate during an elution is altered with time. Initially a fast flow rate is used to rapidly remove the large fraction of bound biomolecules with a relatively fast dissociation rate. Then, to elute the remaining smaller fraction of tightly bound biomolecules, the flow rate is reduced and even combined with extended incubations without flow.

In another preferred embodiment, the flow rate is slow and the washing time is reduced so that all possible interacting molecules that bind the target are selected. These initially selected molecules can by amplified and subjected to more stringent, subsequent selections (e.g., selections having a faster flow rate or longer washing times).

In yet another embodiment, the magnetically responsive particles are subject to stringent washing (e.g., “elution conditions” for an extensive time. Then, interacting molecules that remain bound are recovered from the beads. If the interacting molecules are, e.g., display library members, they can amplified from the beads, or their nucleic acid component can be amplified.

Solution Conditions

Solutions can be judiciously chosen. Scopes, supra, provides a general guide on the various properties of compounds that can be added to a solution. A buffering agent can be used to maintain a stable pH. Examples of buffering agents include the “Goods” buffers such as Tris, HEPES, and PIPES. Additional buffering agents that can be used include phosphate, citrate, and glycine.

Various salts can be include to control ionic strength. Exemplary salts include NaCl, KCl, sodium acetate, sodium citrate, potassium acetate, sodium sulfate, ammonium acetate, and ammonium sulfate. Anions can be selected from the Hofmeister series as follows: SCN⁻, CO₄⁻, NO₃⁻, Br⁻, Cl⁻, acetate⁻, SO₄²⁻, and PO₄³⁻. Cations can be selected from: NH₄⁺, K⁺, and Na⁺. Ammonium sulfate can be used to control the hydrophobic effect. High ammonium sulfate conditions can enhance hydrophobic interactions, whereas low ammonium sulfate conditions can weaken them.

Chaotropic agents can be added to disrupt binding interactions or modulate protein stability. Some exemplary chaotropes include guanidinium hydrochloride and urea. Many detergents are also chaotropic.

Exemplary detergents include sodium dodecyl sulfate (SDS), NP-40, Tween, and non-ionic detergents. Examples of non-ionic detergents include n-octylglucoside, n-dodecylglucoside, n-dodecylmaltoside, octanoyl-N-methylglucamide, decanoyl-N-methylglucamide, Triton® X-100, Triton® X-114, isotridecypoly(ethylene glycol ether)n, 3-[(3-cholamidopropyl)dimethylamminio]-1-propane sulfonate (CHAPS), and 3-[(3-cholamidopropyl)dimethylamminio]-2-hydroxy-1-propane sulfonate (CHAPSO). It can be advantageous to use detergents at a concentration below their critical micelle concentration (CMC).

The redox potential of solutions can also be controlled, e.g., using reducing or oxidizing agents. Exemplary reducing agents include β-mercaptoethanol and dithiothreitol (DTT). Oxidation can be controlled, e.g., by adding known concentrations of oxidized and reduced glutathione.

Solutions can also include a protein or cell-stabilizing reagent, e.g., glycerol. For some applications, e.g., for nucleic acids, organic solvents such as acetonitrile, DMSO, DMF, and formamide can be used.

Solutions can also include divalent cations, e.g., Mg₂⁺, Ca²⁺, and Mn²⁺; metals, e.g., Ni, Zn, Fe; and chelators such as EDTA.

Solutions for applications using cells, e.g., living cells, can include various nutrients and growth factors.

Solutions for applications using enzymes or for the selection of enzymes can include an enzyme substrate or co-factor, e.g., NAD, ATP, GTP, deoxynucleotides, and ribonucleotides.

Applications

The apparati and methods described herein can be utilized for a variety of applications and likewise adapted to any particular appropriate application. Some exemplary methods are described herein. These include: screening a display library, refining a display library, isolating a biomolecule, isolating a cell or cell population, modifying, e.g., chemically modifying, a magnetically responsive particle, and isolating a catalyst.

Generally, a sample is contacted to magnetically responsive particles that have an attached target. The particles are washed in a flow chamber in accordance with the methods described herein. Then, the fraction of the sample that is removed or retained by the wash is provided. In analytical and screening procedures, the fraction can subject to analysis or processing. For example, eluted proteins can be analyzed by mass spectroscopy. Eluted cells, nucleic acids, and display library members can be amplified. For preparative procedures, the fraction of interest may be at a sufficient concentration and/or of sufficient purity to use in downstream applications.

Samples. The sample, frequently a complex mixture, can vary depending on the application. Non-limiting examples of samples are samples that include members of a display library; a population of cells, e.g., tissue cells, blood cells, or microorganisms; a population of proteins, e.g., serum proteins, a cell extract, or in vitro synthesized proteins; a population of nucleic acids, and a population of organic molecules (e.g., non-polymeric organic molecules). Samples can obtained from a natural source or from an artificial, e.g., recombinant or synthetic, source.

In a preferred embodiment, the sample is a display library described below.

In another preferred embodiment, component molecules of the sample are labeled with a detectable substance, e.g., prior to contact with the target. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; an example of a suitable prosthetic group is biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In one embodiment, the sample is homogenous, e.g., a single species, or a combination of a small number of species, e.g., two, three, or about ten species. Such samples can be used in combination with the methods described herein, e.g., to determine if one of the species binds to a target attached to the particles, or in the case of a small number of species, if one species competes with another for binding to the target.

Targets. Generally, any molecular species can be used as a target. In some embodiment, more than one species is used as a target, e.g., a sample is exposed to a plurality of targets. The target can be of a small molecule (e.g., a small organic or inorganic molecule), a polypeptide, a nucleic acid, cells, and so forth.

One class of targets includes polypeptides. Examples of such targets include small peptides (e.g., about 3 to 30 amino acids in length), single polypeptide chains, and multimeric polypeptides (e.g., protein complexes).

A polypeptide target can be modified, e.g., glycosylated, phosphorylated, ubiquitinated, methylated, cleaved, disulfide bonded and so forth. Preferably, the polypeptide has a specific conformation, e.g., a native state or a non-native state. In one embodiment, the polypeptide has more than one specific conformation. For example, prions can adopt more than one conformation. Either the native or the diseased conformation can be a desirable target, e.g., to isolate agents that stabilize the native conformation or that identify or target the diseased conformation.

In some cases, however, the polypeptide is unstructured, e.g., adopts a random coil conformation or lacks a single stable conformation. Agents that bind to an unstructured polypeptide can be used to identify the polypeptide when it is denatured, e.g., in a denaturing SDS-PAGE gel, or to separate unstructured isoforms of the polypeptide for correctly folded isoforms, e.g., in a preparative purification process.

Some exemplary polypeptide targets include: cell surface proteins (e.g., glycosylated surface proteins or hypoglycosylated variants), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins such as integrins, cell-binding proteins (e.g., cell attachment molecules or “CAMs” such as cadherins, selectins, N-CAM, E-CAM, U-CAM, I-CAM and so forth), or a cell surface protein (e.g., of a mammalian cancer cell or a pathogen). In some embodiments, the polypeptide is associated with a disease, e.g., cancer.

The target polypeptide is preferably soluble. For example, soluble domains or fragments of a protein can be used. This option is particularly useful for identifying molecules that bind to transmembrane proteins such as cell surface receptors and retroviral surface proteins.

Cells as Targets. Another class of targets includes cells, e.g., fixed or living cells. The cell can be bound to an antibody that is covalently attached to a paramagnetic particle or indirectly attached (e.g., via another antibody). For example, a biotinylated rabbit anti-mouse Ig antibody is bound to streptavidin paramagnetic beads and a mouse antibody specific for a cell surface protein of interest is bound to the rabbit antibody.

In one embodiment, the cell is a recombinant cell, e.g., a cell transformed with a heterologous nucleic acid that expresses a heterologous gene or that disrupts or alters expression of an endogenous gene. In another embodiment, the cell is a primary culture cell isolated from a subject, e.g., a patient, e.g., a cancer patient. In still another embodiment, the cell is a transformed cell, e.g., a mammalian cell with a cell proliferative disorder, e.g., a neoplastic disorder. In still another embodiment, the cell is the cell of a pathogen, e.g., a microorganism such as a pathogenic bacterium, pathogenic fungus, or a pathogenic protist (e.g., a Plasmodium cell) or a cell derived from a multicellular pathogen.

Cells can be treated, e.g., at a particular stage of the washing step. The treatment can be a drug or an inducer of a heterologous promoter-subject gene construct. The treatment can cause a change in cell behavior, morphology, and so forth. Molecules that dissociate from the cells upon treatment are collected and analyzed.

Examples of cells include, a cancer cell, a hematopoietic cell, BalI cells, primary culture cells, malignant cells, neuronal cells, embryonic cells, placental cells, and non-mammalian cells (e.g., bacterial cells, fungal cells, plant cells) and so forth. Cancer cells, for example, are attached to magnetically responsive particles using an antibody specific for a marker on the cell surface, e.g., CD19 or a cell-surface cancer-specific antigen.

In a preferred embodiment, the cells are recombinant cells. The cells can be transformed with a plasmid that expresses (e.g., under control of an inducible or constitutive promoter) a cell-surface protein of interest. The plasmid can also express a marker protein, e.g., for use in binding the transformed cell to a magnetically responsive particle. In another embodiment, the cells express an intracellular protein, e.g., an oncogene, transcription factor, or cell-signaling protein. The intracellular protein can alter cell behavior or the repertoire of molecules on the cell surface. In still another embodiment, the cells are treated (e.g., using a drug or genetic alteration) to alter the rate of endocytosis, pinocytosis, exocytosis, and/or cell secretion.

Nucleic acid targets. Additional exemplary targets include nucleic acids, e.g., double-stranded, single-stranded, and partially double-stranded DNA such as a site in a regulatory region, a site in a coding region, a tertiary structure e.g., a G-quartet or a telomere; RNA, e.g., double-stranded RNA, single-stranded RNA, e.g., an RNAi, a ribozyme; or combinations thereof. For example, a double stranded nucleic acid that includes a site can be used to identify a DNA-binding domain that binds to that site. The DNA-binding domain can be used in cells to regulate genes that are operably linked to the site. For example, the apparatus can be used to identify a multi-domain zinc finger protein that binds a target site.

Still more exemplary targets include organic molecules. In one embodiment, the organic molecules are transition state analogues and can be used to select for catalysts that stabilize a transition state structure similar to the structure of the analogue. In another embodiment, the organic molecules are suicide substrates that covalently attach to catalysts as a result of the catalyzed reaction.

A target can be a drug, e.g., a drug for which a ligand is required in order to improve purification of the drug, e.g., from a chemical reaction, a bioreactor, a media, milk, or a cell extract. The drug can include a peptide, e.g., a polypeptide or a non-peptide functionality.

Some exemplary targets include: cell surface proteins (e.g., glycosylated surface proteins or hypoglycosylated variants), cancer-associated proteins, cytokines, chemokines, peptide hormones, neurotransmitters, cell surface receptors (e.g., cell surface receptor kinases, seven transmembrane receptors, virus receptors and co-receptors, extracellular matrix binding proteins, cell-binding proteins, antigens of pathogens (e.g., bacterial antigens, malarial antigens, and so forth).

More specific examples include: integrins, cell attachment molecules or “CAMs” such as cadherins, selections, N-CAM, E-CAM, U-CAM, I-CAM and so forth); proteases, e.g., subtilisin, trypsin, chymotrypsin; a plasminogen activator, such as urokinase or human tissue-type plasminogen activator (t-PA); bombesin; factor IX, thrombin; CD-4; CD-19; CD20; platelet-derived growth factor; insulin-like growth factor-I and -II; nerve growth factor; fibroblast growth factor (e.g., aFGF and bFGF); epidermal growth factor (EGF); transforming growth factor (TGF, e.g., TGF-α and TGF-β); insulin-like growth factor binding proteins; erythropoietin; thrombopoietin; mucins; human serum albumin; growth hormone (e.g., human growth hormone); proinsulin, insulin A-chain insulin B-chain; parathyroid hormone; thyroid stimulating hormone; thyroxine; follicle stimulating hormone; calcitonin; atrial natriuretic peptides A, B or C; leutinizing hormone; glucagon; factor VIII; hemopoietic growth factor; tumor necrosis factor (e.g., TNF-α and TNF-β); enkephalinase; mullerian-inhibiting substance; gonadotropin-associated peptide; tissue factor protein; inhibin; activin; vascular endothelial growth factor; receptors for hormones or growth factors; protein A or D; rheumatoid factors; osteoinductive factors; an interferon, e.g., interferon-α,β,γ; colony stimulating factors (CSFs), e.g., M-CSF, GM-CSF, and G-CSF; interleukins (ILs), e.g., IL-1, IL-2, IL-3, IL-4, etc.; decay accelerating factor; immunoglobulin (constant or variable domains); and fragments of any of the above-listed polypeptides. In some embodiments, the target is associated with a disease, e.g., cancer.

Other targets may be relevant to biotechnological applications, e.g., to generate molecules useful for the laboratory. For example, streptavidin, green fluorescent protein, or a nucleic acid polymerase can be a target.

System for Apparatus Control

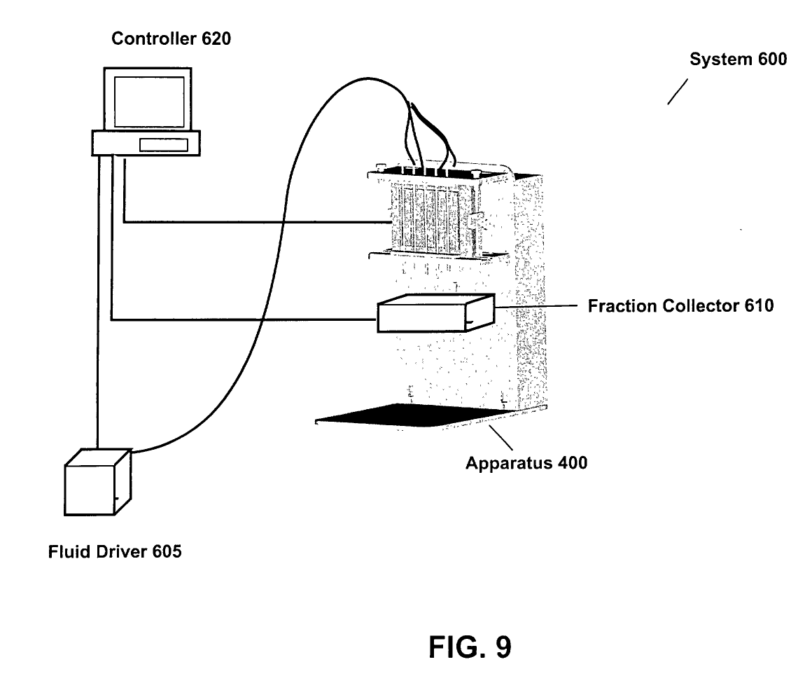

In one embodiment, the washing system 600 is configured to allow flexibility and minimal operator “hands-on” time. Referring now to the example in FIG. 9, the system 600 can include the apparatus 400, a controlled fluid driver 605, various sensors, a fraction collector 610, and a central controller 620. In this embodiment, the apparatus includes multiple capillary flow chambers.

The central controller 620 can be a circuit or programmable device. Typically the controller is a computer such as a PC running Windows®, e.g., Windows 1998® (Microsoft Corp., Redmond Wash.) operating system. The computer is networked or directly connected to other components of the system. The computer can include or be linked to a video monitor, sound speaker, printer, and a device for storing information (e.g. internal RAM, a read-write CD-Rom, flash memory, or a database server).

The computer can include a graphical user interface that has, for example, three modes of operation: The three modes can include: a programming mode; run time mode; and data analysis mode.

Programming-Mode. To facilitate the automated washing of magnetic particles, the controller 620 can communicate with a user using a graphical user interface displayed on a console. The interface can include icons and other tools to facilitate the programming of a washing method.

For example, the interface can display preset values, slider bars, and pop-up question window for the user's convenience. Using such controls or other queries, the interface can obtain information about which capillary flow chambers in the apparatus are to be controlled; the composition, volume, and flow rate of solutions (or gradients thereof) for washing non-specifically or weakly bound materials and for elution; and the size, frequency, and timing of fraction collection. The interface can also obtain parameters to determine the frequency, duration, and speed of the magnetic bead displacement steps. Exemplary parameters are listed in Table 2.

TABLE 2
Exemplary Parameters

| Parameter | Exemplary Value |
|---|---|
| Time between displacement cycles | 5 ms-10 s |
| Number of displacements | 0.1-1 min⁻¹ |
| (e.g. number of rotations of the cam) | |

Run-Time Mode. In run time mode, the controller 620 issues pre-programmed instructions to regulate the fluid driver 605 to deliver fluid to specified capillaries at a specified flow rate or to arrest fluid flow. The pre-programmed instruction can be based on parameters set in the programming mode. The controller 620 also coordinates movement of the magnets 120 and 122 in the apparatus. As described above, the magnets are not moved while liquid is flowing, but when the flow is arrested.

The controller 620 can include a console that displays one or more parameters from the interfaced sensors. The parameters can be graphed with respect to time. In addition, the controller 620 can monitor the parameters. For example, if pressure exceeds a certain level, a sound alarm is generated and flow is stopped. In another example, if the UV-absorbance of the effluent is sufficiently low for a sustained time interval, the controller 620 can activate a program module, e.g., a module that directs elution of material from the flow chamber.

The controller 620 can also be responsive to user commands, e.g., to activate the elution module, or more specifically to activate the fraction collector, e.g., to collect samples of the wash and particular the elution phases.

Data Analysis Mode. The controller can also access information stored during a previous run-time in order to enable a user to analyze or otherwise access the information. An interface can be provided that displays the information to the user, e.g., graphically. For example, the interface can display a graph of UV-absorbance in the effluent line with respect to time. The interface can also display basic information about the run, e.g., the nature of the target, magnetic particles, buffers, and sequence of programmed events. The interface can also coordinate information generated from post-processing of a selection to the run-time data, e.g., in order to enable a user to assess the success of a run.

Computer Systems. The controller 620, its instructions, and other instruction sets described herein may be implemented as programs executing on programmable machines such as mobile or stationary computers, and similar devices that each include a processor, a storage medium readable by the processor, and one or more output devices. Each program may be implemented in a high level procedural or object oriented programming language to communicate with a machine system. Some merely illustrative examples of computer languages include C, Java, and Visual Basic. Each such program may be stored on a medium that is readable by a general or special purpose programmable machine for configuring and operating the processor.

The computer system can be connected to an internal or external network. For example, the computer system can receive requests from a remotely located client system, e.g., using HTTP, HTTPS, or XML protocols. The requests specify a pre-determined program or can detail a sequence of events, i.e., a custom program.

Robotics. The system can control a robot, e.g., a device with a robotic arm and optional sensors, to physically retrieve collection vessels, e.g., a microtitre plate that holds multiple collected fractions from an elution step. The robot can respond to signals from the computer system by moving the microtitre plate to a deck or a conveyance system. The plate can be directed to a post-processing station such as a thermocyclers, an incubator, a storage area (e.g., a plate hotel), or a fluid handling system. A variety of post-processing manipulations are described herein.

Display Libraries

A display library is a collection of entities; each entity includes an accessible polypeptide component and a recoverable component that encodes or identifies the peptide component. The polypeptide component can be of any length, e.g. from three amino acids to over 300 amino acids.

Members of a display library that interact with a target can be identified using the apparati and methods described herein.

Typically, paramagnetic particles that have an attached target are mixed with members of a display library in a tube prior to being placed in the capillary flow chamber. The tube can be rotated gently, e.g. at a temperature between 1° C. and 42° C. for a desired period of tube. Optionally, the particles are washed by immobilizing the particles in the tube in order to remove the majority of the unbound library members prior to placing the particles in the capillary. However, this step can also be omitted in order to prevent dehydration of the particles or protein denaturation due to surface tension effects of aspirating the solution in the tube.

The contents, or an aliquot of the contents, can be placed in a new capillary, which is then attached to an apparatus, e.g., the apparatus of FIG. 4. The first magnetic field is applied to immobilize the paramagnetic particles within the flow chamber. Then, the capillary is attached tubing that delivers liquid from a fluid reservoir. In one embodiment, the tubing is attached to the bottom of the capillary in order direct fluid flow upwards though the capillary.

Optionally, fresh buffer can be manually pipetted into the flow chamber in order to flush out the unbound display library members prior to attaching the tubing.

Preferably, members of the display library never come in contact with tubing or fittings. The configurations that avoid such contact reduce contamination within a given session and during subsequent sessions (e.g., a separate selection). Selections of display library members are particularly vulnerable to contamination as each selection round can include an amplification step that amplifies contaminating library members that do not bind the target as desired. If display library members non-specifically adhere in a supply line that leads into the flow chamber, such non-specific library members can trickle into fractions of interest, e.g., elution fractions.

In a typical selection process, the capillary is washed with a buffer that removes non-specifically and weakly bound library members. The washing process includes cycles of buffer flow, arrest, and agitation as described above. Buffer emerging from the top of the capillary is directed by an effluent line to waste. The amount of time required to wash non-specifically and weakly bound library members can be determined empirically, e.g., by assaying fractions of the effluent for display library members during previous selections, or by monitoring the effluent actively, e.g., for UV-absorbing material or radioactivity (if the library is spiked with radiolabeled members).

After washing non-specifically and weakly bound members, the particles are immobilized, flow is arrested, and the elution solution is directly drawn into the capillary flow chamber, e.g., by placing a container that includes the elution solution directly under the capillary flow chamber. The particles are agitated. Then, the container is removed, and a collection tube is placed under the flow chamber. The flow is reversed to deliver the elution solution and any eluted library members into the collection tube.

A fraction collector or a collection vessel is placed directly under the capillary to collect eluted library members. The collection vessel can be provided with a solution prior to elution. For example, the solution can include a high concentration of a buffering agent in order to neutralize acid, e.g., for an acid elution. In another example, the solution includes glycerol to stabilize eluted material during cryopreservation.

Once configured, the elution solution is flowed through the capillary. As described herein, the elution can include frequent, rapid cycles of agitation. A variety of elution methods can be used, e.g., including step elutions, gradient elutions, e.g., using linearly increasing pH or ionic strength, or the elution method described above for “Off-Rate Selection.”

Eluted library members can be characterized or amplified. For example, the eluted members as a pool can be amplified as appropriate for the format and applied to another selection process. In another example, the eluted members are individually isolated, stored, characterized and/or sequenced. Each individual member is characterized to assess the binding affinity of its polypeptide component to the target. An automated high-throughput ELISA and DNA sequencing system can be used to individually characterize all members of the eluted pool.

A variety of formats can be used for display. The following are some examples.

Phage Display. One format utilizes viruses, particularly bacteriophages. This format is termed “phage display.” The peptide component is typically covalently linked to a bacteriophage coat protein. The linkage results form translation of a nucleic acid encoding the peptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 00/70023; WO 92/18619; WO 91/17271; WO 92/20791; WO 92/15679; WO 93/01288; WO 92/01047; WO 92/09690; WO 90/02809; WO 00/70023; Fuchs et al. (1991) Bio/Technology 9:1370-1372; Hay et al (1992) Hum Antibod Hybridomas 3:81-85; Huse et al. (1989) Science 246:1275-1281; Griffiths et al (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

The terms “bacteriophage library member” and “phage” encompass members of both types of libraries. The term “bacteriophage particle” refers to a particle formed of bacteriophage coat proteins that packages a nucleic acid. The packaged nucleic acid can be a modified bacteriophage genome or a phagemid, e.g., a nucleic acid that includes a bacteriophage origin of replication but lacks essential phage genes and cannot propagate in E. coli without help from “helper phage” or phage genes supplied in trans.

Phage display systems have been developed for filamentous phage (phage fl, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshmand et al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In a preferred embodiment, the fusion is to a domain of the gene III protein, e.g., the anchor domain or “stump.”

The valency of the peptide component can also be controlled. Cloning of the sequence encoding the peptide component into the complete phage genome results in multivariant display since all replicates of the gene III protein are fused to the peptide component. For reduced valency, a phagemid system can be utilized. In this system, the nucleic acid encoding the peptide component fused to gene III is provided on a plasmid, typically of length less than 700 nucleotides. The plasmid includes a phage origin of replication so that the plasmid is incorporated into bacteriophage particles when bacterial cells bearing the plasmid are infected with helper phage, e.g. M13K01. The helper phage provides an intact copy of gene III and other phage genes required for phage replication and assembly. The helper phage has a defective origin such that is the helper phage genome is not efficiently incorporated into phage particles relative to the plasmid that has a wild type origin.

Bacteriophage displaying the peptide component can be grown and harvested using standard phage preparatory methods, e.g. PEG precipitation from growth media.

After selection of individual display phages, the nucleic acid encoding the selected peptide components, by infecting cells using the selected phages. Individual colonies or plaques can be picked, the nucleic acid isolated and sequenced.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and U.S. Provisional Patent Application No. 60/326,320, filed Oct. 1, 2001.

In one embodiment, variegate nucleic acid sequences are cloned into a vector for yeast display. The cloning joins the variegated sequence with a domain (or complete) yeast cell surface protein, e.g., Aga2, Aga1, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg²⁺ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35.

Other Display Formats. Yet another display format is a non-biological display in which the polypeptide component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Scaffolds. Scaffolds for display can include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and beat shock proteins.

Appropriate criteria for evaluating a scaffolding domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffolding domain is a small, stable protein domains, e.g., a protein of less than 100, 70, 50, 40 or 30 amino acids. The domain may include one or more disulfide bonds or may chelate a metal, e.g., zinc.

Examples of small scaffolding domains include: Kunitz domains (58 amino acids, 3 disulfide bonds), Cucurbida maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), Streptococcal G IgG-binding domain (35 amino acids, no disulfide bonds) and small intracellular signaling domains such as SH2, SH3, and EVH domains. Generally, any modular domain, intracellular or extracellular, can be used.

Another useful type of scaffolding domain is the immunoglobulin (Ig) and Ig superfamily domain. An Ig domain refers to a domain from the variable or constant domain of immunoglobulin molecules. An Ig superfamily domain refers to a domain that has a three-dimensional structure related to an Ig domain, but is from a non-immunoglobulin molecule. Ig domains and Ig superfamily domains typically contains two β-sheets formed of about seven β-strands, and a conserved disulphide bond (see, e.g., Williams and Barclay 1988 Ann. Rev Immunol. 6:381-405). Proteins that include domains of the Ig superfamily domains include CD4, platelet derived growth factor receptor (PDGFR), and intercellular adhesion molecule (ICAM).

A preferred embodiment of Ig scaffolds is an antibody, particularly an antigen-binding fragment of an antibody. The term “antibody,” as used herein, refers to an immunoglobulin molecule or an antigen-binding portion thereof. A typical antibody includes two heavy (H) chain variable regions (abbreviated herein as VH), and two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Each VH and VL is composed of three CDR's and four FRs, arranged from amino-terminus to carboxy-terminus in the following order: FR1, CDR1, FR2, CDR2, FR3, CDR3, FR4.

An antibody can also include a constant region as part of a light or heavy chain. Light chains can include a kappa or lambda constant region gene at the COOH—terminus. Heavy chains can include, for example, a gamma constant region (IgG1, IgG2, IgG3, IgG4; encoding about 330 amino acids).

The term “antigen-binding fragment” of an antibody (or simply “antibody portion,” or “fragment”), as used herein, refers to one or more fragments of a full-length antibody that retain the ability to specifically bind to a target. Examples of antigen-binding fragments include, but are not limited to: (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody.

Display technology is particularly useful in order to produce antibodies that bind to self-antigens, e.g., proteins that are recognized as a self by an organism's immune system. Human antibodies that recognize human self-antigens can be used as therapeutics. Since the constant and framework regions of the antibody are human, such therapeutic antibodies may avoid themselves being recognized and targeted as antigens. Further, the constant regions are optimized to recruit effector functions of the human immune system.

Antibody therapeutics can be modified, e.g., to attach to a toxin, e.g., a polypeptide toxin (e.g., ricin or diphtheria toxin or active fragment hereof), a radioactive nucleus, or an imaging agent (e.g. a radioactive, enzymatic, or an NMR contrast agent).

Display technology can also be used to obtain ligands, e.g., antibody ligands, particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-clution agent, e.g., to capture in a wash solution dissociating display library members that are not specific to the target.

Iterative Selection. In one preferred embodiment, display library technology is used in an iterative mode. A first display library is used to identify one or more ligands for a target. These identified ligands are then mutated to form a second display library. Higher affinity ligands are then selected from the second library, e.g., by using higher stringency or more competitive binding and washing conditions.

Numerous techniques can be used to mutate the identified ligands. These techniques include: error-prone PCR (Leung et al. (1989) Technique 1:11-15), recombination, DNA shuffling using random cleavage (Stemmer (1994) Nature 389-391), RACHITT™ (Coco et al. (2001) Nature Biotech. 19:354), site-directed mutagenesis (Zooler et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J 13:3245).

If, for example, the identified ligands are antibodies, then mutagenesis can be directed to the CDR regions of the heavy or light chains. Further, mutagenesis can be directed to framework regions near or adjacent to the CDRs. Likewise, if the identified ligands are enzymes, mutagenesis can be directed to the vicinity of the active site.

Nucleic Acid Aptamers

Random pools of nucleic acid sequences, both DNA and RNA, can be used as a rich source of artificial ligands and catalysts (see, e.g., Ellington and Szostak (1990) Nature 346:818; and (1992) Nature 355:850; and Tuerk and Gold ((1990) Science 249:505 and (1991) J. Mol. Biol. 222:739; U.S. Pat. No. 5,910,408). Such artificial nucleic acid are termed aptamers. Generally, synthetic oligonucleotides are used to assemble pools of random nucleic acid sequences. The sequences can include a constant region or tag which can serve as a primer binding site. The pools are exposed to magnetic particles that have an attached target. The target can be an intended ligand or a transition state analog. Nucleic acids in the pool that bind the target are selected using the methods and apparati described herein. Eluted nucleic acids are amplified. After amplification, the eluted nucleic acid can be used in subsequent rounds of selection or can be characterized.

To evolve catalytic nucleic acids, the target can be a transition state analog or a suicide substrate, e.g., a substrate that reacts with a potential catalyst and covalent attaches to it as a result of the catalyzed reaction. Negative Selection A sample can be contacted first to a non-target molecule, e.g., a molecule related to the target, but yet distinct. For example, in the case of polypeptides and nucleic acids, the non-target and the target molecule can be at least 30%, 50%, 75%, 80%, 90%, or 95% identical to each other. The non-target and target molecule can be identical, but can have different conformations or modifications (e.g., a post-translational modification for polypeptide; a methylation or base adduct for nucleic acid). In one example, the target is a complex of at least two polypeptides (see Brekken et al. (2001) J Control Release. 74:173-81 for an example of an antibody that specifically recognizes a complex), and the non-targets are the component polypeptides in their uncomplexed state. Members of the sample that do not bind the non-target can be collected and used for subsequent selections for binding to the target molecule or even for subsequent negative selections. This procedure allows for the identification of members that bind to the target, but not the non-target.

In one embodiment, the non-target is a constant region, e.g., a peptide tag, purification handle, or attachment moiety that is present during the selection of the target molecule.

The negative selection method is useful, e.g., from modifying a display library prior to selection for binding to the target.

Isolation of Cells Using Magnetic Beads

The apparati described herein can be used to isolate cells, e.g., specific cell types, or to deplete specific cells from a sample. The isolation procedure can be used for preparative or analytical purposes (e.g., diagnostics). For example, non-limiting examples of cells that can be isolated include fetal nucleated cells, stem cells, tumor cells, lymphoid cells, and cell-based display library cells.

Mononuclear cells such as T cells, monocytes, B cells, and NK cells can be isolated from whole blood, bone marrow, and buffy coat. Buffy coat is the layer of white cells that forms between red cells and plasma after the centrifugation of anti-coagulated blood. Specific antibodies are attached to magnetically responsive particles, e.g., using protein A or protein G as a bridging entity. The particles are bound to a mixture of cells, e.g., prior to or after disposing the particles in the flow chamber. Methods can also be modified such that the agitation of the particles in the flow chamber is particularly gentle, e.g., to minimize shear forces.

Useful antibodies for isolating cells on paramagnetic particles include, e.g., anti-CD3, anti-CD4, anti-CD5 and anti-CD8 specific for cytotoxic T lymphocytes, anti-CD12, anti-CD19 and anti-CD20 specific for B cells; anti-CD 14 specific for monocytes; anti-CD 16 and anti-CD56 specific for natural killer cells; anti-CD41 for platelets; anti-CD31 (PECAM-1); anti-CD34 for hematopoietic progenitor cells. Antibodies that specifically recognize tumor cells can be used to isolate such cells. Such antibodies include antibodies that recognize hypoglycosylated MUCI (see, e.g., de Haard et al. (1999) J Biol Chem. 274:18218-30), Her2/Neu, and EpCAM.

Binding agents other than antibodies can also be used to isolate cells. Examples of such binding agents include lectins (e.g., ricin, wheat germ agglutinin, and soy bean agglutinin), growth factors, cytokines, and extracellular matrix molecules.

Cells that are isolated can be cultured and/or analyzed. For example, mRNA can be harvested from the isolated cells and then profiled using a nucleic acid microarray, e.g., as described in Golub et al. (1999) Science 286:531-537. Information from the profile can be used, for example, to identify genes whose regulation is altered in the cell relative to a reference profile, to diagnose a disorder, or to classify the cell. In another example, the isolated cells are labeled, e.g., using specific antibodies and then profiled using Fluorescence-Activated Cell Sorting (FACS). In yet another example, protein is extracted from the cells or from a fraction thereof and analyzed, e.g., using an array of probes that can characterize a protein sample or using mass spectroscopy (see below).

In a preferred embodiment, the cells that are isolated are contacted with a display library, e.g., to identify members that encode high affinity ligands that are specific for the cells.

Processing and Analysis

Fractions obtained from the flow chamber, e.g., from solution leaving the chamber during an elution step, can be processed or analyzed using any appropriate method. Some exemplary methods are described below in the context of eluted fractions. However, they can be applied to any material, e.g., control samples and fractions collected from a wash to remove non-specifically and weakly bound molecules.

In a preferred embodiment, the fractions are obtained from the flow chamber and processed automatically or semi-automatically. For example, the robotic arms, microtitre plate holders, decks, and automatic fluid handlers can be configured as a system to automatically process eluted fractions. Events related to processing can be tracked, e.g., using a computer system. Sample processors and other instruments, e.g., fluorimeters, mass spectrometers, DNA sequencers, thermal cyclers, and BIAcores, can be located “in-line” with the particle washing apparatus.

Amplification. Nucleic acids and cells that are eluted can be amplified. For example, eluted cells can be cultured and grown. If the cells can grow as individual colonies, then after growth, individual colonies can be picked and deposited in the well of an indexed microtitre plate. Each individual clone can then be characterized.

Nucleic acids can be amplified by any appropriate nucleic acid amplification technique. Some exemplary nucleic acid amplification techniques include: the polymerase chain reaction (PCR; Saiki, et al. (1985) Science 230, 1350-1354); transcription-based methods (see, e.g., U.S. Pat. Nos. 6,066,457; 6,132,997; 5,716,785; Sarkar et al., Science (1989) 244: 331-34; Stofler et al., Science (1988) 239: 491); NASBA (see, e.g., U.S. Pat. Nos. 5,130,238; 5,409,818; and 5,554,517); rolling circle amplification (RCA; see, e.g., U.S. Pat. Nos. 5,854,033 and 6,143,495) and strand displacement amplification (SDA; see, e.g., U.S. Pat. Nos. 5,455,166 and 5,624,825).

Amplified nucleic acids can be cloned, e.g., into an expression vector or general cloning vector. Amplified nucleic acids can also be sequenced, e.g., using a high-throughput sequencing device. Further, e.g., as described for selected nucleic acid aptamers, amplified nucleic acids can be used as a sample for additional rounds of selection.

MS (Mass Spectroscopy). Molecules in the eluted fractions can be analyzed by mass spectroscopy, e.g., MALDI-TOF (Matrix-assisted laser desorption-ionization time-of-flight) or electro-spray MS. For example, the eluted fractions can be digested to completion using a protease that recognizes a specific amino acid or site, e.g., trypsin, chymotrypsin, elastase, and papain. The proteolyzed sample is combined with a matrix and a solvent and dried onto a MS plate. The plate is then inserted into as MALDI-TOF mass spectrometer, which excites specific spots on the plate in order to ionize the sample (see e.g., U.S. Pat. No. 6,281,493). The ions are separated according to their mass-to-charge ratio by measuring the time it takes the ions to travel to a detector. The measurement provides a very accurate determination of molecular weight for each proteolyzed fragment in the analyzed fraction. Using a computer system that can access a database of amino acid sequences that might be present in the sample, the identity of the polypeptides in the analyzed fraction can frequently be inferred. Further, post-translation modification can also be identified.

Protein Arrays. Nucleic acids or polypeptides in a fraction to be analyzed can be contacted to a polypeptide array. Methods of producing polypeptide arrays are described in the art, e.g., in De Wildt et al (2000) Nature Biotech. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; and WO 99/51773A1. The fraction can be labeled and then contacted to the array to identify addresses of the array to which the fraction binds. For example, the array can be an array of antibodies, e.g., as described in De Wildt, supra. Information about the extent of binding at each address of the array can be stored as a profile. Profiles can be analyzed, for example, by cluster analysis, in order to compare addresses of the array across multiple fractions or to characterize fractions.

Nucleic Acid Arrays. A fraction can be analyzed to identify nucleic acids present in the fraction, e.g., using a nucleic acid array. For example, the fraction can include cells, and the nucleic acid array can be used to identify genes expressed by the cells in the fraction. In another example, the fraction includes members of a display library of cDNAs. The array can include addresses with probes for multiple cDNA and after hybridization can indicate which cDNAs displayed by the library are present in the fraction.

An nucleic acid array can be constructed by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In vitro Functional Assays. A molecule in an eluted fraction can be further characterized for a functional activity, e.g., for an in vitro activity. One exemplary in vitro assay is an assay for catalysis of a chemical reaction. Substrate for the reaction is supplied in the presence and absence of the molecule and product formation is measured. The measurement reflects that catalytic activity in the assay. The catalytic agent in the assay can be the molecule itself or a given enzyme. In the case wherein the catalytic agent is a given enzyme, the presence of the molecule can enhance or inhibit the catalytic efficiency of the enzyme, e.g., the molecule is an activator or inhibitor of the enzyme.

A molecule in an eluted fraction can be also characterized for a functional activity, e.g., for its ability to affect cell differentiation or cell proliferation in culture (or in vivo or ex vivo). Numerous cell culture assays for differentiation and proliferation are known in the art. Some examples are as follows:

Assays for embryonic stem cell differentiation (which will identify, among others, proteins that influence embryonic differentiation hematopoiesis) include, e.g., those described in: Johansson et al. (1995) Cellular Biology 15:141-151; Keller et al. (1993) Molecular and Cellular Biology 13:473-486; McClanahan et al. (1993) Blood 81:2903-2915.

Assays for lymphocyte survival/apoptosis (which will identify, among others, proteins that prevent apoptosis after superantigen induction and proteins that regulate lymphocyte homeostasis) include, e.g., those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233 243, 1991; Zacharchuk, Journal of Immunology 145:4037 4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

ELISA. The binding interaction of an eluted molecule for a target can be analyzed using an ELISA assay. For example, the molecule is contacted to a microtitre plate whose bottom surface has been coated with the target, e.g., a limiting amount of the target. The molecule is contacted to the plate. The plate is washed with buffer to remove non-specifically bound molecules. Then the amount of the molecule bound to the plate is determined by probing the plate with an antibody specific to the molecule. The antibody is linked to an enzyme such as alkaline phosphatase, which produces a calorimetric product when appropriate substrates are provided. In the case of a display library member, the antibody can recognize a region that is constant among all display library members, e.g., for a phage display library member, a major phage coat protein.

Homogeneous Assays. After a molecule is identified in a fraction, its binding interaction with a target can be analyzed using a homogenous assay, i.e., after all components of the assay are added, additional fluid manipulations are not required. For example, fluorescence energy transfer (FET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). By titrating the amount of the first or second binding molecule, a binding curve can be generated to estimate the equilibrium binding constant.

Surface Plasmon Resonance (SPR). After a molecule is identified in a fraction, its binding interaction with a target can be analyzed using SPR. For example, after sequencing of a display library member present in a sample, and optionally verified, e.g., by ELISA, the displayed polypeptide can be produced in quantity and assayed for binding the target using SPR. SPR or real-time Biomolecular Interaction Analysis (BIA) detects biospecific interactions in real time, without labeling any of the interactants (e.g., BlAcore). Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander, S. and Urbaniczky, C. (1991) Anal. Chem. 63:2338-2345; Szabo et al. (1995) Curr. Opin. Struct. Biol. 5:699-705 and http://www.biacore.com/.

Information from SPR can be used to provide an accurate and quantitative measure of the equilibrium dissociation constant (K_{d}), and kinetic parameters, including Kₒₙ and K_{off}, for the binding of a biomolecule to a target. Such data can be used to compare different biomolecules. For example, proteins selected from a display library can be compared to identify individuals that have high affinity for the target or that have a slow K_{off}. This information can also be used to develop structure-activity relationship (SAR) if the biomolecules are related. For example, if the proteins are all mutated variants of a single parental antibody or a set of known parental antibodies, variant amino acids at given positions can be identified that correlate with particular binding parameters, e.g., high affinity and slow K_{off}.

Additional methods for measuring binding affinities include fluorescence polarization (FP) (see, e.g., U.S. Pat. No. 5,800,989), nuclear magnetic resonance (NMR), and binding titrations (e.g., using fluorescence energy transfer).

Storage. Fractions that emerge from the flow chamber can be stored, e.g., at 4° C., −20° C., and −80° C. If the fraction is to be frozen, the fraction can include a cryoprotectant, e.g., a polyol such as glycerol or sorbitol, sucrose, or DMSO. If the fraction is a stable to lyophilization, the fraction can be frozen and then air-dried under vacuum.

Fermentor for Cell Growth and Selection

A fermentor is a vessel which is configured to support the growth of a biologic.

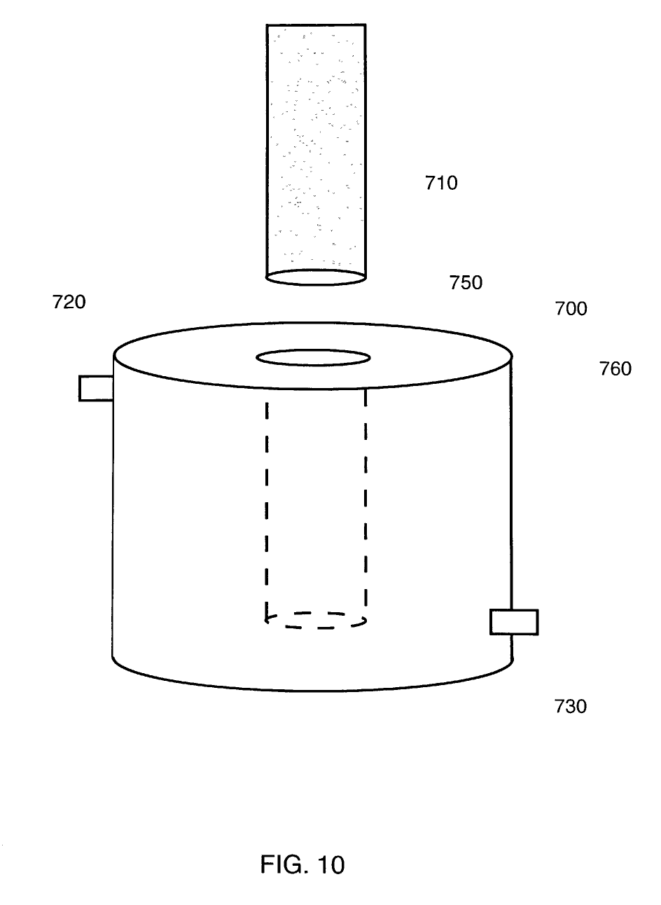

Referring to the example in FIG. 10, a flow chamber 700 for cell growth and selection is depicted. The flow chamber includes an inlet 720 for flowing in a medium for cell growth and wash solutions, and an outlet 730 for disposal of the medium and for collecting samples of interest, e.g., from a elution. Of course, in another embodiment, the port 730 can be the inlet, and the port 720, the outlet. The flow chamber 700 includes an aperture 750 for a sheath 760. A magnetic field inducer 710 can be inserted into the flow chamber in order to capture magnetically responsive particles in a zone surrounding the sheath 760.

The flow chamber 700 further includes a stirrer (not shown; preferably non-magnetic), e.g., which is rotated in order to circulate the growth medium and wash solutions through the chamber. When the magnetic field inducer 710 is removed, the stirrer is used to agitate the magnetically responsive particles.

The flow chamber 700 can also include an oxygenation system, a temperature controller, and glucose, pH and CO₂ monitors (not shown).

The flow chamber can be used to select members of a cell-display library. For example, a yeast cell display library that displays immunoglobulin molecules is mixed in the flow chamber with magnetically responsive particles that include a target compound. The magnetic field inducer 710 is inserted into the sheath 760 to capture the particles. PBS (phosphate buffered-saline) or another wash solution is flushed through the chamber. Flow is arrested, and the magnetic field is released to agitate the particles. Then the magnetic field is reapplied. This wash method can be repeated for a number of cycles.

After washing, media, e.g., YEPD or minimal media, can be flowed through the chamber under conditions that allow the cells to grow and multiply. After an interval sufficient for a desired number of cell divisions elapses, the wash method can be repeated, e.g., with a more stringent wash solution.

The system can include repeatedly washing and growing of the cells as required. The use of the flow chamber for cell growth and multiplication allows for immediate amplification of cells that bind to the magnetically responsive particles and can obviate the need for some of the external manipulations that are required for multiple selection cycles. As a result the cells are amplified in the presence of the target which is attached to the magnetically responsive particles. Similarly other replicable entities can be grown in the flow chamber (or any flow chamber herein) so that the replicable entities can be amplified in the presence of a target compound. For example, phage display library members can be amplified by addition of host cells (and helper phage for phagemids) for sufficient time that a burst is formed.

Fractions of the solutions (e.g., media or wash solutions) that are emerging from the flow chamber can be collected. In particular, fractions can be taken during any wash step, and during the most stringent washing step, i.e., an elution step. The fraction can be plated, e.g., onto agar plates with yeast media, in order to form colonies from the individual cells that are present in the fraction. The fraction can also be diluted into liquid media to amplify the cells outside of the growth chamber.

In another embodiment, the flow chamber has a single port, e.g., a flask with an upper opening that is sealable. Fluid is removed and provided from the single port. In still other embodiments, the flow chamber has multiple ports, e.g., three or more ports.

Some implementations are described in more detail through the following practical examples. However, it should be noted that these examples are not limiting.

### EXAMPLE

Two types of yeast cells displaying Fab fragments were separated using the apparatus with a capillary flow chamber. The first yeast cell displays a Fab (PHI) that binds to the MUC1 antigen and is genetically marked as TRP1⁺ leu2⁻. The second yeast cell displays a Fab (α-strep) that binds to streptavidin and is genetically marked as trpl⁻ LEU2⁺. The two types of yeast cells were incubated with 100 μl of streptavidin beads for one hour.

The mixture was disposed in a 200 μl capillary tube in the apparatus. A flow rate of 1 ml/min was used to deliver fluid to the tube. The magnet was agitated every 10 seconds. During agitation, the fluid flow was reduced to 0 ml/min. The tube was washed for 13 minutes with 2% MPBBS, then with PBS for 2 minutes.

Beads and any bound yeast cells were recovered from the capillary and plated onto minimal media plates lacking either leucine or tryptophan. As shown in Table 3, the procedure resulted in at least a 76,000 fold enrichment for yeast cells displaying the Fab (α-strep).

TABLE 3

| Clone | Input | Output | Ratio | Enrichment |
|---|---|---|---|---|
| PH1 -leu | 1.9 · 10⁹ | 1.2 · 10³ | 6.3 · 10⁻⁷ | 76190 |
| F2 -trp | 2.1 · 10⁵ | 1.0 · 10⁴ | 4.8 · 19⁻² | |

### EXAMPLE

The following procedure was used to assess enrichment of phage display library members “panned” against Ball1 cells captured with CD19 Pan-B paramagnetic beads.

The following test phages were used for selection. The specific binding phage that was expected to bind the target and the non-specific phage were mixed in a ratio such that the non-specific phage (fd-tet, tetracyclin resistant) was in a 2000 fold excess to the specific binding phage (ampicillin resistant) (1×10¹¹:5×10⁷). As noted, the two phages were genetically marked with different antibiotic resistant markers. A small aliquot was used to perform titration (input).

First, all relevant materials were blocked. An U-bottom 96-wells plate (Costar) was blocked with 2%Marvel PBS (MBPS). 1×10⁶ Ball1 cells (B-cell line) per selection were blocked in 200 μl 2%MPBS. 10 μl CD19 pan-B paramagnetic beads (Dynal M-450) were blocked in 200 μl 2%MPBS (beads were washed 1 cycle with 2%MPBS) and the capillary was blocked with 2%MPBS. The phage mix was also blocked by adding 100 μl 4%MPBS/0.1% NaN₃ to 100 μl phage. Blocking time was 0.5-1 hour for the 96 wells plate, cells, beads, capillary and phages.

After blocking, the anti-CD 19 containing paramagnetic beads were drawn to one site of an Eppendorf tube by a special magnet (Dynal®). The supernatant was removed. The paramagnetic beads were resuspended with a 200 μl solution containing the blocked Ball1 cells and gently rotated for 15 min. at room temperature to bind the beads to the cells. The cell-bead complex was washed for 1 cycle to remove unbound cells.

Then 200 μl pre-blocked phage mix was added to the cell-bead complex (again by use of the magnet to remove the previous supernatant from the Eppendorf tube) and incubated for 1 hour at room temperature with gentle shaking. After incubation of phages with cell-beads complex, the mixture was transferred to the pre-blocked 96 wells plate and introduced into the capillary of the capillary washing device (see, e.g., FIGS. 4 to 8) (the capillary are located in 96 wells plate format to enable easy loading of the capillary) and washed for 15 minutes with 2%MPBS/0.05%NaN₃ with a flow rate of 115 μl/min. During the wash, the magnets were moved from a first to a second position every 10 seconds (with 90 translations in the course of 15 min. washing time). During the wash, the pump kept running maintaining a constant flow rate of 115 μl/min (i.e., no fluid flow arrest was used). After the 15 minute 2%MPBS/0.05NaN₃ wash, the beads were captured on one side of the capillary by applying the magnet to the one side of the capillary. The washing buffer was exchanged with PBS and then an additional wash of 5 minutes was performed under similar conditions.

The bound phages were eluted by filling the capillary with 200 μl TEA 100 mM (triethylamine). The TEA was introduced to the capillary with an air bubble between the washing buffer and the TEA solution. During this process, the beads were again arrested at one side by applying the magnet to one side of the capillary. During the elution the magnet was moved from one side to the other every minute for a total elution time of 5 minutes. Magnetic forces on the beads were eliminated by moving the magnet in a position whereby the capillary was in the middle of the permanent magnets to collect the eluted materials in a tube containing 100 μl IM Tris-HCL pH 7.4 that neutralizes the TEA.

The mixture was briefly mixed. Cell debris and remaining beads were spun down by a short centrifugation step of 5 min. at 14000 RPM. The supernatant was transferred to a new tube (named: output). A dilution series of input and output samples was made and used to infect Tg1 cells growing exponentially (OD600=0.5). Infection was for 30 min at 37° C. without agitation. Dilutions were plated on agar plates containing ampicillin or tetracycline in order to score the population size of non-specific and specific binding phages. After growth, colony forming units were counted on the agar plates.

TABLE 4

| Clone | Input | Output | Ratio | Enrichment |
|---|---|---|---|---|
| Tet | 1 · 10¹¹ | 3.8 · 10⁵ | 3.8 · 10⁻⁶ | 208 |
| F9 Anti-CD20 | 5.2 · 10⁷ | 4.1 · 10⁴ | 7.9 · 10 E⁻⁴ | |
| Tet | 1.4 · 10¹¹ | 2.8 · 10⁵ | 2.0 · 10⁻⁶ | 375 |
| F9 Anti-CD20 | 1.4 · 10⁷ | 1.8 · 10⁴ | 7.5 · 10 E⁻⁴ | |

The results indicate amino acid at least a 200 fold enrichment of specific binding phage when selected against cells using the capillary washing device.

### EXAMPLE

The following procedure demonstrates the selection of certain phage display library members using a capillary flow chamber under different flow conditions. In particular, the method was used to discriminate between stronger binders and weaker binders to a cell surface antigen that is present on the surface of human umbilical cord endothelial cells (HUVEC). The procedure used six characterized phage isolates that each display a Fab fused to the gene III bacteriophage surface protein. These six phage each display a Fab that binds to an antigen (Target X) expressed on the surface of HUVEC. The phage are distinguishable from each other by DNA footprinting and sequencing.

For the procedure, the six HUVEC-binding Fab-displaying phage clones were mixed in equal amounts (10⁷ phage/clone) with 10¹⁰ fd-tet-dog phage (non-specific phage) to form a mixture with seven different types of phage (six HUVEC-binding, and one fd-tet-dog phage). The enrichment of the six Fab displaying phage versus the background Fd-tet-Dog1 phage was determined by counting the number of recovered phage before and after selection procedures. The Fab displaying phage are, when infected into E. coli cells, resistant to ampicillin, while the Fd-Tet-Dog1 phage when infected into E. coli cells, are resistant to tetracycline. A small aliquot of the starting mixtures was used to perform a titration (Input).

All relevant materials were blocked prior to the procedure. An U-bottom 96-well plate (Costar) was blocked with 2% Marvel PBS (MPBS). 1×10⁶ HUVEC per selection were blocked in 200 μl 2% MPBS/10% FCS/0.01% NaN₃. 10 μl anti-CD31 Endothelial cell paramagnetic beads (Dynal M-450) were blocked in 200 μl 2% MPBS (beads were washed 1 cycle with 2% MPBS) and the capillary was blocked with 2% MPBS. The phage mix was also blocked by adding 100 μl 4% MPBS/10% FCS/0.01% NaN₃ to 100 μl phage. Blocking time was 0.5 hour for the 96 wells plate, cells, beads, capillary and phages.

After blocking, the anti-CD31 containing paramagnetic beads were drawn to one site of an Eppendorf tube by a magnet (Dynal®). The supernatant was removed. The paramagnetic beads were resuspended in 200 μl solution containing the blocked HUVEC and gently rotated for 30 min. at room temperature to bind the beads to the cells. The cell-bead complex was washed for two cycles to remove unbound cells. Then 200 μl pre-blocked phage mix was added to the cell-bead complex (again using the magnet to remove the previous supernatant from the Eppendorf tube) and incubated for one hour at room temperature with gently shaking. After incubation of phages with cell-bead complex, the mixture was transferred to the pre-blocked 96 well plate and then introduced into the capillary of the capillary washing device (the capillary are located in a 96 wells plate format to enable easy loading of the capillary) and washed for 10 minutes with 2% MPBS/10% FCS/0.01% NaN₃ with a flow rate of 100 μl/min for Procedure 1 and 200 μl/min for Procedure II.

Capillary washing action was controlled by entering parameters into a graphical interface that accepted values (time in minutes and seconds) for the following:

Interval Time:Time between one movement of magnet from the first position to the second position.

Pump Delay Time:Delay time of pump within Interval Time

Total Run Time:Total time of washing in capillary

Motor on Time:Time that the motor for moving the magnets is switched on (magnet speed dependent)

In these implementations, the interval time was set for 10 seconds, the pump delay time for 0 seconds, total run time for 10 minutes, and motor on time for 1 second.

The following parameters can be independently set using software that is connected to the flow chamber and fluid pump. The interface also displaced the process status (e.g., “Motor=OFF|Pump=Off|Direction=Left|Nr of Processes=0,” for an idle state). The interface can also include buttons for initiating actions, e.g., Start, Stop, Load, Save.

After the 10 minute 2% MPBS/10% FCS/0.01% NaN₃ wash, the beads were captured on one site of the capillary by applying the magnet to one site of the capillary. The washing buffer was exchanged with PBS. Then, an additional wash of 5 minutes was performed under similar conditions.

The bound phage were eluted by filling the capillary with 200 μl TEA 100 mM (triethylamine). The TEA was introduced into the capillary with an air bubble between the washing buffer and the TEA solution. During this process, the beads were again arrested at one site by applying the magnet to one side of the capillary. The bound phages were eluted for 5 minutes under similar conditions (without flow).

Magnetic forces on the beads were eliminated by moving the magnet in a central position whereby the capillary was in the middle of two permanent magnets to collect the eluted materials in a new tube. For this elution the direction of the pump was reversed The mixture was briefly mixed. Cell debris and remaining beads were spun down by a short centrifugation step of two min. at 14000 RPM. The supernatant was transferred to a new tube containing 100 μl 1M Tris-HCL pH 7.4 that neutralizes the TEA (named: output). A dilution series of input and output samples was made and used to infect TG1 E. Coli cells growing exponentially (OD600=0.5). Infection was performed for 30 min. at 37° C. without agitation. Dilutions were plated on agar plates containing either ampicillin or tetracycline in order to score the population of non-specific (fd-Tet-Dog1) and specific binding phages (Fab displaying phages binding to target X). After growth, colony-forming units were counted on the agar plates. In addition 41 colonies per selection from the ampicillin input and output plates were used to perform a DNA fingerprint analysis on by digesting the PCR products (colony PCR) with BstNI restriction enzyme. This was done to identify the unique clones binding to Target X. Fingerprint patterns were compared before and after selection to estimate the frequency and distribution of the 6 clones after washing with certain stringency. The six different clones are expected to have different affinities for the Target X and therefore enrichment under different conditions are expected to differ.

Two selections with capillary washing were performed as described above but with increasing washing stringency. The effect of the flow rate (stringency of washing was examined) by analysis of enrichments and DNA fingerprint patterns.

TABLE 5
Procedure I (flow rate 100 μl/ml)

| Clone | Input | Output | Ratio | Enrichment |
|---|---|---|---|---|
| Tet | 3.9 × 10¹⁰ | 1.7 × 10⁵ | 4.4 × 10⁻⁶ | |
| Amp | 2.3 × 10⁷ | 1.3 × 10⁴ | 5.7 × 10⁻⁴ | 130 |

TABLE 6
Procedure II (flow rate 200 μl/ml)

| Clone | Input | Output | Ratio | Enrichment |
|---|---|---|---|---|
| Tet | 1.5 × 10¹⁰ | 1.7 × 10⁴ | 1.1 × 10⁻⁶ | |
| Amp | 5.0 × 10⁷ | 1.4 × 10⁴ | 2.8 × 10⁻⁴ | 255 |

TABLE 7
DNA Fingerprint analysis of clones after washing at 100 μl/ml

| | Frequency | Frequency |
|---|---|---|
| | (Input) | (Output) |
| Clone | Procedure I | Procedure I |
| A2 | 8 | 1 |
| A12 | 4 | 12 |
| A6 | 8 | 19 |
| A8 | 3 | 0 |
| C6 | 8 | 3 |
| G3 | 6 | 6 |

TABLE 8
DNA fingerprint analysis of clones after washing at 200 μl/ml

| | Frequency | Frequency |
|---|---|---|
| | (Input) | (Output) |
| Clone | Procedure II | Procedure II |
| A2 | 2 | 4 |
| A12 | 3 | 8 |
| A6 | 6 | 18 |
| A8 | 10 | 3 |
| C6 | 9 | 0 |
| G3 | 8 | 8 |

To correlate the frequency found back after selection (DNA fingerprint data) with avidity/affinity of clones a standard phage ELISA was performed with dilutions of phage from each of these 6 clones. The amount of phage used 1e10, 1e9, 1e8, 1e7, 1e6 and 1e5. Under the conditions used for ELISA the phage clone giving the lowest phage titre at half OD₄₅₀ max. is expected to bind strongest to the target.

An ELISA plate was coated with the target protein (5 μg/ml) overnight at 4° C. The next day the plate was washed 2 cycles with PBS/0.1% Tween and one cycle with PBS. Dilutions of phage were prepared and both phage and ELISA plate were blocked for 30 min. with 2% Marvel/PBS. After blocking plate was washed 2 cycles with PBS/0.1% Tween and one cycle with PBS. Phage dilutions were added to corresponding wells and incubated 1.5 hours shaking at RT. After phage incubation the ELISA plate was washed 5 cycles with PBS/0.01% Tween and one cycle with PBS. Anti M13-HRP (1:5000 diluted in 2% Marvel/PBS) was added to each well (100 μl/well) and incubated one hour shaking at RT. ELISA plate was again washed 5 cycles with PBS/0.01% Tween and 1 cycle with PBS. 100 μl TMB solution was added to each well. After 10 min the reaction was stopped by adding 50 μl 2N H₂SO₄. Color was measured at 450 nm in plate reader. See Table 9.

TABLE 9

| | Clone | | | | | | |
|---|---|---|---|---|---|---|---|
| Phage Added (CFU) | A2 | A12 | A6 | A8 | C6 | G3 | neg. |
| 1.00E+10 | 2.039 | 1.894 | 1.829 | 1.867 | 1.786 | 1.905 | 0.098 |
| 1.00E+09 | 2.008 | 1.982 | 2.099 | 1.894 | 1.748 | 1.621 | 0.081 |
| 1.00E+08 | 1.83 | 1.791 | 1.537 | 1.596 | 1.589 | 1.578 | 0.055 |
| 1.00E+07 | 0.865 | 0.795 | 0.704 | 0.597 | 0.821 | 0.709 | 0.053 |
| 1.00E+06 | 0.181 | 0.132 | 0.148 | 0.182 | 0.175 | 0.211 | 0.051 |
| 1.00E+05 | 0.082 | 0.088 | 0.076 | 0.077 | 0.067 | 0.074 | 0.05 |

Values are OD450 from ELISAs for bound phage.

Conclusions. The results from Table 5 and 6 show that the enrichments of specific phage binding to Target X versus background phage is higher for the higher flow rate condition used (255 versus 130).

The DNA fingerprint analysis of clones before and after selection shown in Table 7 and 8 shows that under both experimental conditions certain clones are selected over other clones comparing input versus output. See for example clone A12 in Table 7 and 8. Table 7 and 8 also show that differences in flow conditions do have an influence in the output of the selections. For example clone C6 (Table 7, 100 μl/ml flow) that is found as input 8 times is found back 3 times in the output while the same clone C6 under high flow conditions (200 μl/ml, Table 8) is not found back at all in the output (input C6; Table 8). This demonstrates that under the high flow conditions some low affinity clones are selected against whereas some high affinity clones are enriched.

Finally to identify if these results match with the affinity (and avidity) of the Fabs displayed binding to Target X, a specificity ELISA with different concentrations of phage was performed.

The results in Table 9 show that there are some differences in binding to the recombinant Target X. For example clone A12 enriched strongly under both selection conditions shows the strongest binding to the Target X.

The fact that some strong binding clones (shown in Table 9) are not enriched dramatically (for example clone A2) could be related to the difference in epitope recognition and the conformation of the protein expressed on HUVEC cells.

In a second type of procedure, the above procedure was performed, except no elution of the phages was performed. Instead, the whole mixture after washing (without elution) was used to infect E. coli cells.

### EXAMPLE

The following is an example of a method of amplifying a display library member in the presence of a target compound; this method does not require a magnetic bead washing apparatus. Approximately 100 μl of Dynal Sv coated magnetic beads (Dynal M280) were blocked with 500 μl of 2% milk in PBS (MPBS) for 30 minutes. Following a wash step to remove the excess milk, 3·10¹¹ Fab-displaying phage from a Fab-fragment phage display library diluted in MPBS was incubated with the blocked Sv beads in a total volume of 500 ul for 1 hour at room temperature. The Sv magnetic beads were collected and the unbound phage were removed by aspiration. The beads were washed three times in lx PBS followed by the addition of 5001 μl of XL1 Blue-MRF′ cells (Stratagene) at an OD₆₀₀ of 0.50 in 2×YT. The mixture was incubated at 37° C. for 15 minutes at which time 5 μl of 100 mM IPTG was added to achieve a final concentration of 1 mM IPTG. At 20 minutes, the bacteria were transferred to a 30° C. air shaker for an additional 25 minutes for a total incubation time of 45 minutes. The bacteria were removed and the beads were washed three times with 500 μl of 0.01% Tween-20 PBS. An additional 500 μl of XL1Blue-MRF′ cells were supplemented to the beads and the process of incubating and washing was repeated for a total of 3 rounds. The 500 μl of phage infected bacteria from each round were titered on ampicillin-containing plates as well as grown overnight in 10 mL of 2×YT containing 1 mM IPTG at 30° C. The resulting phage were purified by standard PEG precipitation.

Three parallel experiments were carried out. In the first, the temperature was held at 37° C. for 20 minutes and then dropped to 30° C. for 25 minutes. In the second, the temperature of incubation was held constant at 37° C. for 45 minutes. In the third, the temperature was held constant at 30° C.

The titres of the various rounds are shown in Table 10. In the headings, “37×20+30×25” denotes the experiment in which the first 20 minutes of incubation was at 37° C. and the final 25 minutes was at 30° C., “37×45” denotes the experiment in which 37° C. was used for 45 minutes, and “30×45” denotes the experiment in which 30° C. was used for 45 minutes.

TABLE 10

| | 37 × 20 + 30 × 25 | 37 × 45 | 30 × 45 |
|---|---|---|---|
| cfu round 1/foi | 5.8e7/1.95E−4 | 8e7/2.7E−4 | 5.1e7/1.7E−4 |
| cfu round 2/foi | 5.1e5/0.009 | 1.2e6/0.015 | 7.5e5/0.012 |
| cfu round 3/foi | 7.3e4/0.14 | 5.4e4/0.045 | 4.8e4/0.063 |

### EXAMPLE

The following is an example of a method of amplifying a display library of phagemids in the presence of a target compound; this method does not require a magnetic bead washing apparatus. The method includes the following steps:

1) Mix phagemid library with biotinylated target

2) Capture target and binding phage on Sv beads

3) Wash away non-binding phagemid (cold target can be used for a limited time to elute weak binders; as many washes as needed can be performed)

4) Add F+cells and growth medium

5) Incubate for time TI (between 30 min to 120 minutes, optionally with antibiotic which could be added after time T2 to select for infected cells)

6) Aliquot cells into empty vials (one per target) and plate as round 1

7) Set round counter R=1

8) Into each vial, add helper phage and target on Sv beads (if necessary additional target could be added after the helper phage, burst of phagemid is expected 30-45 minutes after addition of helper phage)

9) At time T3, wash away cells and non-binding phage (optionally can use cold target-wash for a limited time to elute weak binders)

10) Add cells and GM

11) Incubate for time T4 (e.g., 30 min to 120 minutes; probably=T1) (antibiotic can be added after time T5 (probably=T2) to select for infected cells)

12) Make round counter R=R+1

13) Transfer an aliquot of cells to new counter

14) Plate an aliquot of cells for colonies as round “R” (2, 3, . . . )

15) Go back to step 8 as needed.

Other Embodiments

Those skilled in the art will recognize, or be able to ascertain using no more than routine experimentation, many embodiments in addition to the specific embodiments of the invention described herein. Such embodiments are within the following claims.

What is claimed:

1. A method of washing magnetically responsive particles, the method comprising:
flowing liquid through a flow chamber that includes magnetically responsive particles captured in a first zone by a first magnetic field;
dissipating the first magnetic field; and
agitating the magnetically responsive particles.

2. The method of claim 1 further comprising reducing or arresting the liquid flow prior to the dissipating.

3. The method of claim 1 further comprising repeating the flowing through the agitating one or more times.

4. The method of claim 1 wherein the agitating is effected by cyclically re-applying the first magnetic field.

5. The method of claim 1 wherein the agitating is effected by applying a second magnetic field.

6. The method of claim 5 wherein the agitating is effected by alternatively applying the second and first magnetic field.

7. The method of claim 5 wherein the first and second magnetic field are applied by first and second permanent magnets that are coordinately regulated.

8. The method of claim 1 wherein the flow chamber comprises a glass cylinder.

9. The method of claim 7 wherein the first and second magnets are attached to a common frame, which is translated between a first and second position to alternatively apply the first and second magnetic fields.

10. The method of claim 1 wherein the magnetically responsive particles comprise a target.

11. The method of claim 10 wherein the magnetically responsive particles are bound by a first compound that remains bound in the liquid and a second compound, that dissociates in the liquid.

12. The method of claim 1 wherein the liquid comprises media for cell growth.

13. The method of claim 11 wherein the liquid comprises a competing agent that competes with the target for binding of the first compound.

14. The method of claim 1 wherein the magnetically responsive particles are retained in the flow chamber throughout the flowing, dissipating, and agitating.

15. A method comprising:
providing a flow chamber, a first magnetic field inducer, and magnetically responsive particles disposed in the chamber, each particle physically associated with a target;
capturing the magnetically responsive particles in a first magnetic field applied by the first magnetic field inducer in the flow chamber;
flowing a solution through the flow chamber; and
displacing the magnetically responsive particles from the first zone of the flow chamber to one or more other regions of the flow chamber such that the magnetically responsive particles are retained in the flow chamber.

16. The method of claim 15 wherein the displacing comprises altering the position of the first magnetic field inducer relative to the flow chamber.

17. The method of claim 16 wherein the displacing comprises applying a second magnetic field to displace the magnetically responsive particles to a second zone.

18. The method of claim 17 wherein the first magnetic field inducer comprises a permanent magnet.

19. The method of claim 10 wherein the target comprises a cell.

20. The method of claim 10 wherein the target comprises a purified polypeptide.

21. The method of claim 15 wherein the target is bound by a first compound that remains bound in the liquid and a second compound that dissociates in the liquid.

22. The method of claim 21 further comprising isolating the first compound from the second compound.

23. The method of claim 22 wherein the first and second compounds are display library members.

24. The method of claim 15 wherein the particles are displaced in a substantially perpendicular direction relative to the direction of solution flow.

25. A method comprising:
a) disposing in a flow chamber magnetically responsive particles that have a target attached;
b) applying a first magnetic field to the flow chamber;
c) contacting the magnetically responsive particles to a mixture of compounds;
d) flowing liquid through the chamber; and
e) agitating the magnetically responsive particles by alternatively applying the first magnetic field and a second magnetic field.

26. The method of claim 25 wherein c) is effected prior to a).

27. The method of claim 25 wherein c) is effected prior to b).

28. The method of claim 25 wherein the order is sequential.

29. The method of claim 25 wherein the mixture comprises cells

30. The method of claim 25 wherein the mixture comprises a display library.

31. The method of claim 30 wherein the display library is a phage display library.

32. The method of claim 30 wherein the display library is a cell display library.

33. The method of claim 32 wherein the flow chamber is maintained under conditions such that at least some of the cells of the display library divide.

34. The method of claim 25 wherein a programmable processor controls the flow of liquid and application of the first and second magnetic fields.

35. A method comprising:
providing a flow chamber that includes a mixture comprising magnetically responsive particles and members of a display library, the magnetically responsive particles having attached thereto a target compound;
washing at least some of the members from the particles by effecting one or more cycles that comprise (i) flowing a first solution through the flow chamber while the particles are captured by a magnetic field, and (ii) agitating the particles by selectively applying one or more magnetic fields.

36. The method of claim 35, further comprising washing at least others of the members from the particles by effecting one or more cycles that comprise (i) flowing a second solution through the flow chamber while the particles are captured by a magnetic field, and (ii) agitating the particles by selectively applying the magnetic field.

37. The method of claim 36 wherein the first and second solutions have different ionic strengths.

38. The method of claim 36 wherein the first and second solutions have different pH.

39. The method of claim 36 wherein the second solution comprises a protease.

40. The method of claim 36 wherein the first solution maintains disulfide bonds and the second solution reduces disulfide bonds.

41. The method of claim 36 wherein the second solution comprises a competing agent.

42. The method of claim 36 further comprising amplifying at least some of the display library members in the second solution and repeating the method.

43. The method of claim 36 wherein the first or second solution comprises a medium for cell growth.

44. A method comprising:
providing a flow chamber having an inlet, an outlet and magnetically responsive particles disposed therein;
translating to a first position a frame that includes a first and second magnetic field inducer, wherein the first position locates the first magnetic field inducer such that a first magnetic field is applied to the flow chamber and the second magnetic field inducer such that a second magnetic field is not applied to the flow chamber;
flowing liquid through the flow chamber; and
translating the frame to a second position wherein the second position locates the first magnetic field inducer such that a first magnetic field is not applied to the flow chamber and the second magnetic field inducer such that a second magnetic field is applied to the flow chamber.

45. The method of claim 44 wherein the flow is controlled by a fluid driver, translation of the frame is controlled by a regulator, and the fluid driver and the regulator are in signal communication.

46. The method of claim 44 wherein the flow is controlled by a fluid driver, translation of the frame is controlled by a regulator, the fluid driver and the regulator are controlled by a programmable processor.

47. An apparatus comprising:
a flow chamber having an inlet and an outlet; and
at least a first magnetic field inducer, wherein the apparatus is configured such that a first and second magnetic field can be selectively applied in the flow chamber.

48. An apparatus comprising:
a flow chamber having an inlet and an outlet and including magnetically responsive particles; and
at least a first magnetic field inducer that selectively applies a first magnetic field, wherein the magnetically responsive particles are retained in the flow chamber in the absence of the first magnetic field.

49. An apparatus comprising:
a flow chamber having an inlet and an outlet; and
first and second magnetic field inducers, each magnetic field inducer being controllable to selectively generate apply a magnetic field in a zone of the flow chamber.

50. The apparatus of claim 48, further comprising a second magnetic field inducer that selectively applies a second magnetic field.

51. The apparatus of claim 48 wherein the first magnetic field inducer is a permanent magnet.

52. The apparatus of claim 50 wherein the first and second magnetic field inducers are coupled such that they alternatively generate a magnetic field in the flow chamber.

53. The apparatus of claim 52 wherein the first and second magnetic field inducers are rigidly connected.

54. The apparatus of claim 48 wherein the flow chamber comprises a glass cylinder having a long diameter less than 2 mm.

55. An apparatus comprising:
a support having a fitting adapted for mounting a flow chamber;
at least a first and second magnetic field inducers;
a translatable frame having attached to the magnetic field inducers; and
an actuator that translates the frame in response to a control signal, wherein the translation moves the magnetic field inducers relative to a flow chamber if mounted.

56. The apparatus of claim 55 wherein the actuator comprises an eccentrically driven cam that is attached to the frame.

57. A system comprising:
the apparatus of claim 48;
a fluid control unit in fluid communication with the flow chamber;
a machine comprising a processor configured to execute instructions, the instructions causing the machine to effect a method comprising:
detecting a user command; and
in response to the command, sending controls to the apparatus and fluid control unit to effect for one or more cycles of
(1) activating flow of liquid by triggering the fluid control unit; and
(2) activating the apparatus to agitate magnetically responsive particles by alternately applying at least a first magnetic field.

58. A method comprising: forming a complex, in a vessel, that includes (a) magnetically responsive particles, (b) a target that is attached or attachable to the particles, and (c) a replicable entity that displays a heterologous protein component; applying a magnetic field that retains the complex in the vessel; removing fluid from the vessel; supplying a solution that supports replication of the replicable entity to the vessel; and replicating the first replicable entity by one or more cycles of replication.

59. A method of selecting a replicable display entity, the method comprising:
a) providing a library of replicable entities that each have a heterologous protein component that is physically attached to the respective replicable entity, wherein each protein component is a member of diverse set of different proteins;
b) contacting replicable entities of the library to a target;
c) performing one or more cycles of:
i) forming replicable entity-immobilized target complexes, each of which includes (1) a replicable entity that binds to the target by its heterologous protein component and (2) the target immobilized to a support;
ii) separating replicable entities that do not bind to the target from the replicable entity-immobilized target complexes,
iii) producing copies of the replicable entities in the presence of the target, the produced copies being replicates of replicable entities that bind to the target; and
d) recovering the nucleic acid encoding the heterologous protein component of one or more produced replicable entities that bind to the target, thereby selecting a nucleic acid that encodes a binding protein for the target.

60. The method of claim 59 wherein the replicable entity is a yeast display cell.

61. The method of claim 59 wherein the replicable entity is a phage display particle.

62. A method of amplifying a replicable display entity, the method comprising:
providing a vessel that comprises a plurality of replicable entities that include a heterologous protein component and a target;
binding a first subset of the replicable entities of the plurality to the target;
separating a second subset of replicable entities of the plurality from the first subset, wherein the replicable entities of the second subset do not bind to the target; and
effecting repeated cycles of replication and solution replacement while members of the first subset are captured by the target.
